(19) United States
(12) Patent Application Publication
(10) Pub. No.: US 2022/0234012 A1
(43) Pub. Date: Jul. 28, 2022

(54) SPARGER ASSEMBLIES FOR A BIOPROCESSING SYSTEM

(71) Applicant: GLOBAL LIFE SCIENCES SOLUTIONS USA LLC,, MARLBOROUGH, MA (US)

(72) Inventors: ANDREAS CASTAN, ALVSJO (SE); RICHARD DAMREN, MARLBOROUGH, MA (US)

(73) Assignee: GLOBAL LIFE SCIENCES SOLUTIONS USA LLC, MARLBOROUGH, MA (US)

(21) Appl. No.: 17/612,640

(22) PCT Filed: May 21, 2020
(86) PCT No.: PCT/EP2020/064216

§ 371 Date: Nov. 19, 2021

Related U.S. Application Data
(60) Provisional application No. 62/850,725, filed on May 21, 2019.

(51) Int. Cl. B01F 23/231; C12M 1/00; C12M 1/04; C12M 1/12; B01F 23/23; B01F 23/233; B01F 27/113; B01F 35/513

(52) U.S. Cl. CPC B01F 23/231231; B01F 2101/44; C12M 23/24; C12M 23/26; C12M 23/28; C12M 25/02; C12M 29/06; B01F 23/231151; B01F 23/23125; B01F 23/231266; B01F 23/238; B01F 23/23362; B01F 27/113; B01F 35/513; C12M 23/14

(57) ABSTRACT

A sparger assembly for a bioprocessing system includes a first layer having a plurality of pores of a first size, and a second layer disposed above the first layer and having a plurality of holes of a second size, the second size being greater than the first size. The pores of the first layer and the holes of the second layer allow for the passage of a sparge gas through the first layer and the second layer.

This application is a national stage of International Application No. PCT/EP2020/064216 filed on May 21, 2020, which claims priority to U.S. Provisional Patent Application No. 62/850,725 filed on May 21, 2019, all of which are hereby incorporated by reference in their entireties.

### BACKGROUND

### Technical Field

Embodiments of the invention relate generally to bioprocessing systems and methods and, more particularly, to impeller and sparger assemblies for single-use bioreactor systems.

### Discussion of Art

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless steel shell or vessel. Use of sterilized disposable bags eliminates time-consuming step of cleaning of the vessel and reduces the chance of contamination. The bag may be positioned within the rigid vessel and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel. Magnetic coupling of the agitator inside the bag, to a drive system or motor external to the bag and/or bioreactor vessel, can eliminate contamination issues, allow for a completely enclosed system, and prevent leakage. Because there is no need to have a drive shaft penetrate the bioreactor vessel wall to mechanically spin the agitator, magnetically coupled systems can also eliminate the need for having seals between the drive shaft and the vessel.

Depending on the fluid being processed, the bioreactor system may include a number of fluid lines and different sensors, probes and ports coupled with the bag for monitoring, analytics, sampling, and liquid transfer. For example, a harvest port is typically located at the bottom of the disposable bag and the vessel, and allows for a harvest line to be connected to the bag for harvesting and draining of the bag. In addition, existing bioreactor systems typically utilize spargers for introducing a controlled amount of a specific gas or combination of gases into the bioreactor. A sparger outputs small gas bubbles into a liquid in order to agitate and/or dissolve the gas into the liquid. The delivery of gas via spargers helps in mixing a substance, maintaining a homogenous environment throughout the interior of the bag, and is sometimes essential for growing cells in a bioreactor. Ideally, the spargers and the agitator are in close proximity to ensure optimal distribution of the gases throughout the container.

One type of known sparger used in many cell culture processes is the drilled hole sparger. This type of sparger is well suited to deliver nominal gas flow through the bioreactor vessel, which is needed to control the partial pressure of carbon dioxide. One drawback, however, is that the size of the holes in the sparger is such that liquid from the vessel/processing environment can leak through the holes back into the gas supply line (particularly when the sparge gas is turned off or decreased). In some situations, the liquid can travel through the gas supply line and potentially reach upstream components such as the mass flow controller, affecting the operation thereof.

In addition to the above-noted drawbacks of existing sparger assemblies, many existing sparger assemblies have fixed pore/hole diameters, which produce a fixed distribution of bubble diameters in the bioreactor vessel. Due to this constraint, a compromise must often be made in the selection of the sparger for use in a given bioreactor, in order to select one sparger that produces a distribution of bubble diameters which is effective over a range of bioreactor operating conditions. Some bioreactor systems allow for the use of different (i.e., multiple) spargers in a single bioreactor in an effort to accommodate a wider range of operating conditions. Both of these options, however, require that sparger selection take place during the design process, prior to fabrication of the bioprocessing bag. This inability to produce different bubble diameter distributions under different sparge gas mass transfer requirements can cause a number of undesirable effects, such as excessive foaming on the surface of the bioreactor.

In connection with the above, high performance bioreactor systems must provide good bulk mixing in combination with efficient gas dispersion in order to achieve a high gas surface area and bubble size distribution, and thus provide high oxygen transfer rates and kLa (the volumetric mass-transfer coefficient that describes the efficiency with which oxygen can be delivered to a bioreactor for a given set of operating conditions) values desired in intensified cell culture and/or microbial applications. Traditional solutions for achieving high kLa values employ multiple impellers mounted on a single shaft. With single-use bioreactors, however, the use of multiple impellers results in a bulky format of the disposable bag, which cannot be collapsed efficiently. Moreover, longer shafts with multiple impellers requires stabilization, which increases the complexity and cost of the vessel and bag design, and renders bag installation more cumbersome and less user friendly.

In view of the above, there is a need for impeller and/or sparger assemblies that provide for increased oxygen transfer rates and kLa values in a bioreactor system to support increased cell culture cell densities. In addition, there is a need for sparger assemblies that prevent or inhibit backflow of liquid from the bioreactor vessel into the sparge gas supply lines, and which enable sparge gas bubble diameter and/or distribution to be selectively adjusted during the cell culturing process.

### BRIEF DESCRIPTION

In an embodiment, a sparger assembly for a bioprocessing system is provided. The sparger assembly includes a first layer having a plurality of pores of a first size, and a second layer disposed above the first layer and having a plurality of holes of a second size, the second size being greater than the first size. The pores of the first layer and the holes of the second layer allow for the passage of a sparge gas through the first layer and the second layer.

In another embodiment of the invention, a bioprocessing system is provided. The bioprocessing system includes a vessel, a flexible bioprocessing bag positionable within the vessel, and a sparger assembly positioned at a bottom of the flexible bioprocessing bag. The sparger assembly includes a first layer having a plurality of pores of a first size, and a second layer disposed above the first layer and having a plurality of holes of a second size, the second size being greater than the first size, wherein the pores of the first layer and the holes of the second layer allow for the passage of a sparge gas through the first layer and the second layer.

In yet another embodiment of the invention, a sparger assembly is provided. The sparger assembly includes a base layer, a dielectric layer disposed above the base layer, a top layer disposed above the dielectric layer, the top layer having an upper surface, at least one electrode in contact with the dielectric layer, and at least one sparge gas opening in at least the hydrophobic layer for facilitating the formation of a bubble of sparge gas on the upper surface of the top layer for introduction of the sparge gas into a bioreactor vessel.

In yet another embodiment, a bioprocessing system is provided. The system includes a vessel, a flexible bioprocessing bag positionable within the vessel, and a sparger assembly positioned at a bottom of the flexible bioprocessing bag. The sparger assembly incudes a base layer, a dielectric layer disposed above the base layer, a top layer disposed above the dielectric layer, the top layer having an upper surface, at least one electrode in contact with the dielectric layer, and at least one sparge gas opening in at least the hydrophobic layer for facilitating the formation of a bubble of sparge gas on the upper surface of the top layer for introduction of the sparge gas into a bioreactor vessel.

In yet another embodiment, a method for bioprocessing is provided. The method includes the steps of positioning a sparger assembly in a bioreactor vessel, the sparger assembly having a base layer, a dielectric layer disposed above the base layer, a top layer disposed above the dielectric layer, the top layer having an upper surface, at least one electrode in contact with the dielectric layer, and at least one sparge gas opening in at least the hydrophobic layer for facilitating the formation of a bubble of sparge gas on the upper surface of the top layer for introduction of the sparge gas into the bioreactor vessel, electrically connecting the at least one electrode to a voltage source, and adjusting a voltage supplied to the at least one electrode to adjust a diameter of the bubble formed on the upper surface of the top layer.

### DRAWINGS

The present invention will be better understood from reading the following description of non-limiting embodiments, with reference to the attached drawings, wherein below:

FIG. 1 is a front elevational view of a bioreactor system according to an embodiment of the invention.

FIG. 2 is a simplified side elevational, cross-sectional view of the bioreactor system of FIG. 1.

FIG. 3 is a perspective view of a sparger assembly for use with the bioreactor system of FIG. 1, according to an embodiment of the invention.

FIG. 4 is a perspective view of a sparger assembly according to another embodiment of the invention.

FIG. 5 is a perspective view of a sparger assembly according to another embodiment of the invention.

FIG. 6 is a perspective view of a sparger assembly according to an embodiment of the invention.

FIG. 7 is a perspective view of a sparger assembly according to an embodiment of the invention.

FIG. 8 is a perspective view of a sparger assembly according to an embodiment of the invention.

FIG. 9 is a perspective view of a sparger assembly according to an embodiment of the invention, shown with an impeller assembly mounted thereon.

FIG. 10 is a top plan view of the sparger assembly of FIG. 9.

FIG. 11 is a perspective view of an impeller assembly for use with the bioreactor system of FIG. 1, according to an embodiment of the invention.

FIG. 12 is a top plan view of the impeller assembly of FIG. 11.

FIG. 13 is a side elevational view of the impeller assembly of FIG. 11.

FIG. 14 is an enlarged, detail view of area A of FIG. 13.

FIG. 15 is a perspective view of an impeller assembly for use with the bioreactor system of FIG. 1, according to another embodiment of the invention.

FIG. 16 is a perspective view of an impeller assembly for use with the bioreactor system of FIG. 1, according to another embodiment of the invention.

FIG. 17 is a perspective view of an impeller assembly for use with the bioreactor system of FIG. 1, according to another embodiment of the invention.

FIG. 18 is a schematic illustration of the impeller assembly of FIG. 17.

FIG. 19 is a perspective view of an impeller assembly for use with the bioreactor system of FIG. 1, according to another embodiment of the invention.

FIG. 20 is a perspective view of an impeller assembly for use with the bioreactor system of FIG. 1, according to another embodiment of the invention.

FIG. 21 is a schematic illustration of an arrangement of apertures in a sparger element/aeration manifold, according to an embodiment of the invention.

FIG. 22 is a top plan view of one arrangement of aeration manifolds of a sparger assembly, according to an embodiment of the invention.

FIG. 23 is a perspective view of a sparger assembly according to another embodiment of the invention.

FIG. 24 is a side elevational view of the sparger assembly of FIG. 23, shown in use in a flexible bioreactor bag.

FIG. 25 is a diagrammatic view of a sparger assembly according to another embodiment of the invention.

FIG. 26 is a diagrammatic view of a sparger assembly according to yet another embodiment of the invention.

FIG. 27 is a diagrammatic view of the sparger assembly of FIG. 26, in an energized state.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems, e.g., chromatography and tangential flow filter systems, and their associated flow paths. As used herein, the term “bag” means a flexible or semi-rigid container or vessel used, for example, as a bioreactor or mixer for the contents within.

As used herein, the term “removably connected” or “removably coupled” means that the aeration manifolds/sparger elements and base plate are connected in such a way as to be easily connected and/or removed to allow for easy user customization of a sparger assembly without special tools. In other words, “removably connected” is an opposite of “permanently connected”.

Embodiments of the invention provide bioreactor systems and sparger assemblies for a bioreactor system. In an embodiment, a sparger assembly for a bioprocessing system includes a base plate and at least one aeration manifold connected to the base plate in spaced vertical relation to the base plate. Each aeration manifold includes at least one inlet for receiving a gas and a plurality of gas outlet openings for delivering the gas to a fluid within the bioprocessing system.

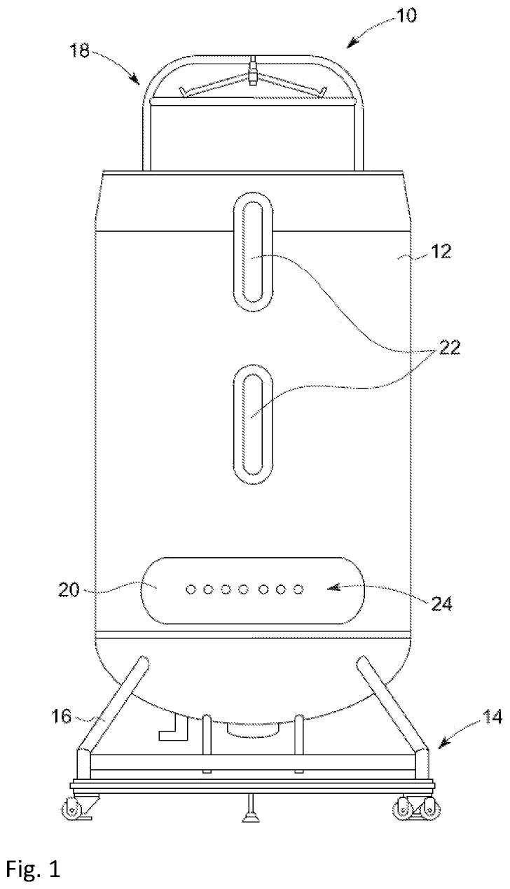

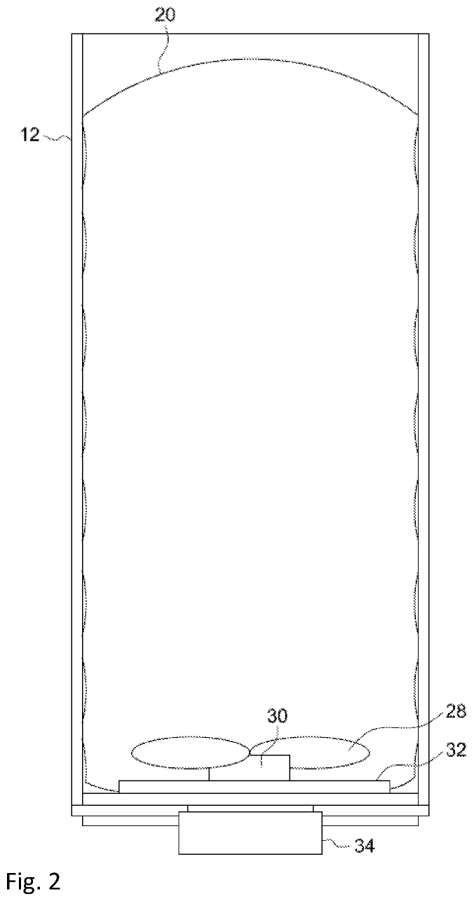

With reference to FIGS. 1 and 2, a bioreactor system 10 according to an embodiment of the invention is illustrated. The bioreactor system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 may be outfitted with a lift assembly 18 that provides support to a single-use, flexible bag 20 disposed within the vessel 12. The vessel 12 can be any shape or size as long as it is capable of supporting a single-use flexible bioreactor bag 20. For example, according to one embodiment of the invention the vessel 12 is capable of accepting and supporting a 10-2000L flexible or collapsible bioprocess bag assembly 20.

The vessel 12 may include one or more sight windows 22, which allows one to view a fluid level within the flexible bag 20, as well as a window 24 positioned at a lower area of the vessel 12. The window 24 allows access to the interior of the vessel 12 for insertion and positioning of various sensors and probes (not shown) within the flexible bag 20, and for connecting one or more fluid lines to the flexible bag 20 for fluids, gases, and the like, to be added or withdrawn from the flexible bag 20. Sensors/probes and controls for monitoring and controlling important process parameters include any one or more, and combinations of: temperature, pressure, pH, dissolved oxygen (DO), dissolved carbon dioxide (pCO2), mixing rate, and gas flow rate, for example.

With specific reference to FIG. 2, a schematic side elevational, cutaway view of the bioreactor system 10 is illustrated. As shown therein, the single-use, flexible bag 20 is disposed within the vessel 12 and restrained thereby. In embodiments, the single-use, flexible bag 20 is formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem^{TM,} Bioclear^{TM} 10 and Bioclear 11 laminates, available from GE Healthcare Life Sciences. Portions of the flexible container can comprise a substantially rigid material such as a rigid polymer, for example, high density polyethylene, metal, or glass. The flexible bag may be supplied pre-sterilized, such as using gamma irradiation.

The flexible bag 20 contains an impeller 28 attached to a magnetic hub 30 at the bottom center of the inside of the bag, which rotates on an impeller plate 32 also positioned on the inside bottom of the bag 20. Together, the impeller 28 and hub 30 (and in some embodiments, the impeller plate 32) form an impeller assembly. A magnetic drive 34 external to the vessel 12 provides the motive force for rotating the magnetic hub 30 and impeller 28 to mix the contents of the flexible bag 20. While FIG. 2 illustrates the use of a magnetically-driven impeller, other types of impellers and drive systems are also possible, including top-driven impellers.

In an embodiment, the impeller plate 32 may be configured as a sparger assembly that is used to introduce a specific gas or air into the fluid within the bag 20 in order to agitate and/or dissolve the air or gas into the fluid. Accordingly, in some embodiments, the impeller and sparger, and the components thereof, form a combined impeller/sparger assembly. In other embodiments, the sparger assembly and the impeller assembly may be separate and/or discrete components. In either implementation, the sparger assembly and the impeller assembly are in close proximity to ensure optimal distribution of gases throughout the bag 20, as discussed in detail hereinafter. As discussed below, it is envisioned that the sparger assembly (which may also serve as an impeller plate supporting the impeller) may take one of various configurations.

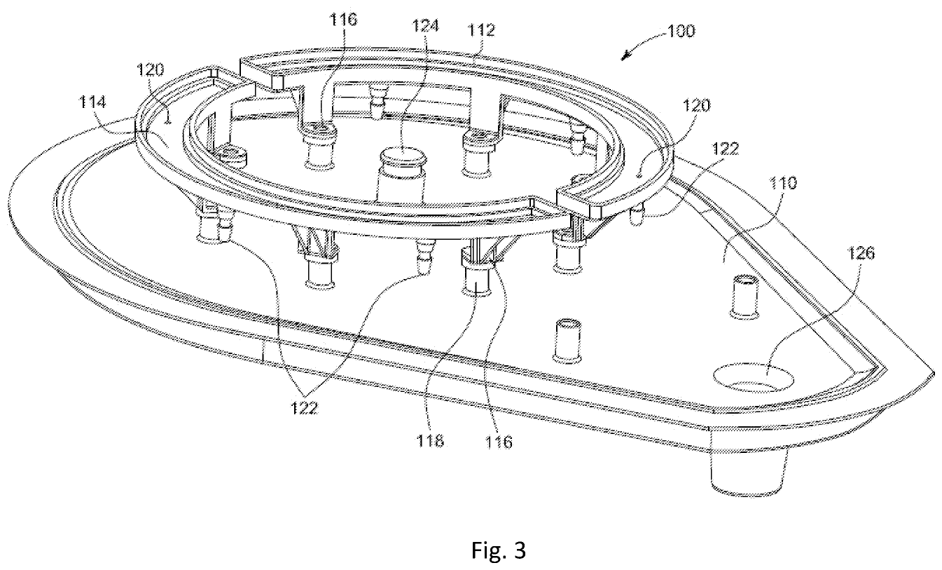

For example, FIG. 3 illustrates one embodiment of a sparger assembly 100 that may be utilized with the flexible bag 20 and bioreactor/bioprocessing system 10. As shown therein, the sparger assembly 100 includes a base plate 110 and a plurality of aeration channels or hollow aeration elements or manifolds 112, 114 connected to the base plate 110. In an embodiment, the aeration manifolds 112, 114 include a plurality of feet 116 that are received on corresponding stand-offs or mounting posts 118 of the base plate 110 such that the aeration manifolds 112, 114 are supported in vertically-spaced relation to (i.e., raised above) the base plate 110. In an embodiment, the aeration manifolds 112, 114 and base plate 110 may be manufactured as an integral, unitary component. In other embodiments, the aeration manifolds 112, 114 may be manufactured as separate components that may be removably coupled to the base plate 110 through a snap fit, clips, screws or other connection means using the feet 116 and posts 118. As shown in FIG. 3, each of the aeration manifolds 112, 114 may be arc-shaped. In an embodiment, as shown in FIG. 3, the manifolds 112, 114 may be semi-circular arcs and include a plurality of gas outlet openings or apertures 120 in a top surface thereof. In an embodiment, the gas outlet openings 120 may be pores in a porous frit. The aeration manifolds 112, 114 may also include one or more tube connectors 122 forming an inlet configured for mating connection with a gas supply line (not shown) for delivering gas to the aeration manifolds 112, 114. In an embodiment, the tube connectors 122 are hose barb connectors, although other connector types known in the art may also be utilized without departing from the broader aspects of the invention.

In an embodiment, the gas outlet openings 120 may all be the same size. In other embodiments, the gas outlet openings 120 of the first aeration manifold 112 may be a different size than the has outlet openings 120 of the second aeration manifold 114. For example, the gas outlet openings 120 of the first aeration manifold 112 may be smaller than the gas outlet openings 120 of the second aeration manifold 114. In such an implementation, therefore, the first aeration manifold 112, with its comparatively small gas outlet openings 120 that produce relatively small gas bubbles, may be utilized to supply oxygen, while the second aeration manifold 114, with its comparatively large gas outlet openings 120 that produce relatively large gas bubbles, is particularly suited for stripping or sweeping out CO2 with air, for example. Where a porous frit is utilized, the openings/pores will not have the same size, however, the various aeration manifolds may have openings with the same or different average size.

With further reference to FIG. 3, the base plate 110 may include a mounting device enabling coupling of an impeller of the bioprocessing system to the sparger assembly in close association with the aeration manifold. In an embodiment, the mounting device is a vertically-extending mounting shaft 124 centrally located between the two arc-shaped manifolds 112, 114. The shaft 124 is configured to receive the magnetic hub (e.g., hub 30) of an impeller (e.g., impeller 28) and to support the impeller in a position where the lower edge of the impeller blades are positioned just proud of the top surface of the manifolds 112, 114. While embodiments described herein disclose the sparger assembly as having a mounting shaft for receiving an impeller assembly, other cooperating mounting arrangements are possible. For example, the sparger assemblies disclosed herein may have a recessed bearing or receiver structure that is configured to receive a shaft fixed to the impeller. Other coupling arrangements are also possible without departing from the broader aspects of the invention and may include any configuration that employs a retention element to couple the impeller and base plate to one another.

In an embodiment, the base plate 110 may further include an aperture 126 or fitting for fluid coupling with drain tubing for draining or harvesting of the contents of the flexible bag 20. Incorporating the impeller mounting shaft 124 and the drain aperture 126 into the base plate 110 facilitates positioning of the flexible bag 20 within the bioreactor vessel 10, as well as facilitates alignment of the magnetic hub 30 with the magnetic drive system and drain port in the flexible bag 20 with the drain tubing connected to the bottom of the bioreactor vessel 20.

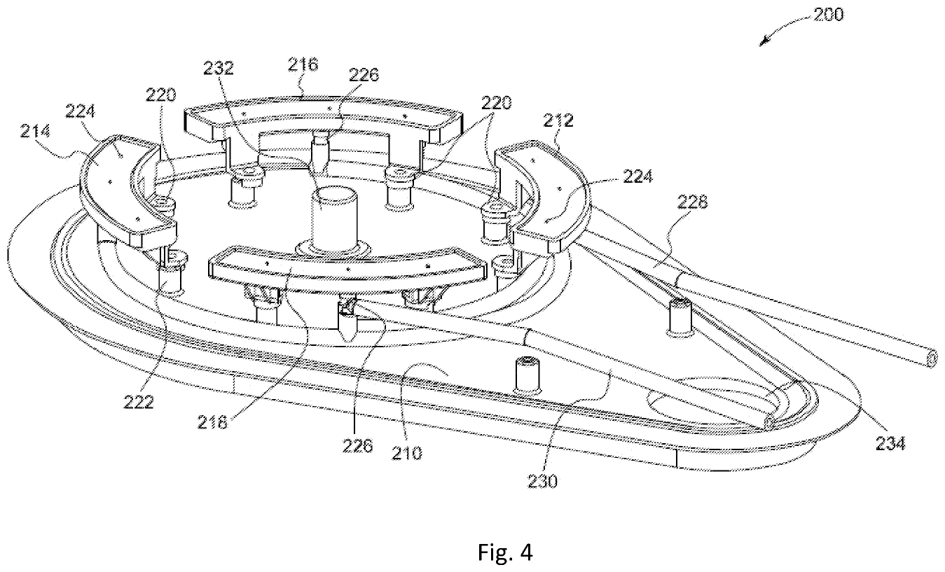

Turning now to FIG. 4, a sparger assembly 200 according to another embodiment of the invention is illustrated. As shown therein, the sparger assembly 200 includes a base plate 210 and a plurality of, namely four, aeration channels or hollow aeration manifolds 212, 214, 216, 218 connected to the base plate 210. In an embodiment, the aeration manifolds 212, 214, 216, 218 include a plurality of feet 220 that are received on corresponding stand-offs or mounting posts 222 of the base plate 210 such that the aeration manifolds are supported in vertically-spaced relation to (i.e., raised above) the base plate 210, as described above. As also described above, the aeration manifolds and base plate may be manufactured as an integral, unitary component, or as separate components that may be removably coupled to the base plate 210 through a snap fit, clips, screws or other connection means using the feet 220 and posts 222.

As shown in FIG. 4, each of the aeration manifolds may be quarter-circular arcs and include a plurality of gas outlet openings or apertures 224 in a top surface thereof. The aeration manifolds 212, 214, 216, 218 may also include one or more tube connectors 226 forming an inlet configured for mating connection with one or more gas supply lines, e.g., lines 228, 230 for delivering gas to the aeration manifolds. In an embodiment, the tube connectors 226 are hose barb connectors, although other connector types known in the art may also be utilized without departing from the broader aspects of the invention.

Similar to the embodiment of FIG. 3, the gas outlet openings 224 of each aeration manifold may be the same size. In other embodiments, the size of the gas outlet openings 224 of at least one of the aeration manifolds may be different from the size of the gas outlet opening s 224 of at least another of the aeration manifolds. For example, in an embodiment, a first pair of opposing aeration manifolds, e.g., aeration manifolds 212, 214 on opposite sides of the circle formed by the arrangement of the manifolds on the base plate 210, may have gas outlet openings 224 of a first size that is different from the size of the gas outlet openings 224 of a second pair of opposing aeration manifolds, e.g., aeration manifolds 216, 218 on opposite sides of the circle formed by the arrangement of the manifolds on the base plate. As disclosed above, the aeration manifolds with the smaller gas outlet openings may be utilized to supply oxygen, while the aeration manifolds with the larger gas outlet openings may be utilized to strip or sweep out CO2, with air, for example.

In yet another embodiment, an immediately adjacent pair of aeration manifolds, e.g., aeration manifolds 212, 216 may have gas outlet openings 224 of a first size, while another immediately adjacent pair of aeration manifolds, e.g., aeration manifolds 214, 218 may have gas outlet openings of a second size, wherein the second size is different from the first size. The configuration of the base plate 210 and aeration manifolds 212, 214, 216, 218, and the selectively removable nature of the aeration manifolds, allows the configuration of the sparger assembly 200 to be easily adjusted according to user preferences. In particular, this design allows for plug-and-play like functionality, enabling a user to mount various combinations of aeration manifolds to the base plate 210 to provide a sparger assembly of various configurations. For example, a user can easily mount three aeration manifolds with smaller gas outlet openings 224 in combination with a single aeration manifold with larger gas outlet openings 224 to increase oxygen delivery to the system, where desired, or mount three aeration manifolds with larger gas outlet openings 224 in combination with a single aeration manifold with smaller gas outlet openings 224 to enhance CO2 removal, without having to adjust rate of gas delivery to the sparger assembly 200.

As discussed above in connection with FIG. 3, the base plate 210 may include a vertically-extending mounting shaft 232 centrally located between the aeration manifolds for receiving an impeller assembly. Moreover, as discussed above, the base plate 210 may include an aperture 234 or fitting for fluid coupling with drain tubing for draining or harvesting of the contents of the flexible bag 20.

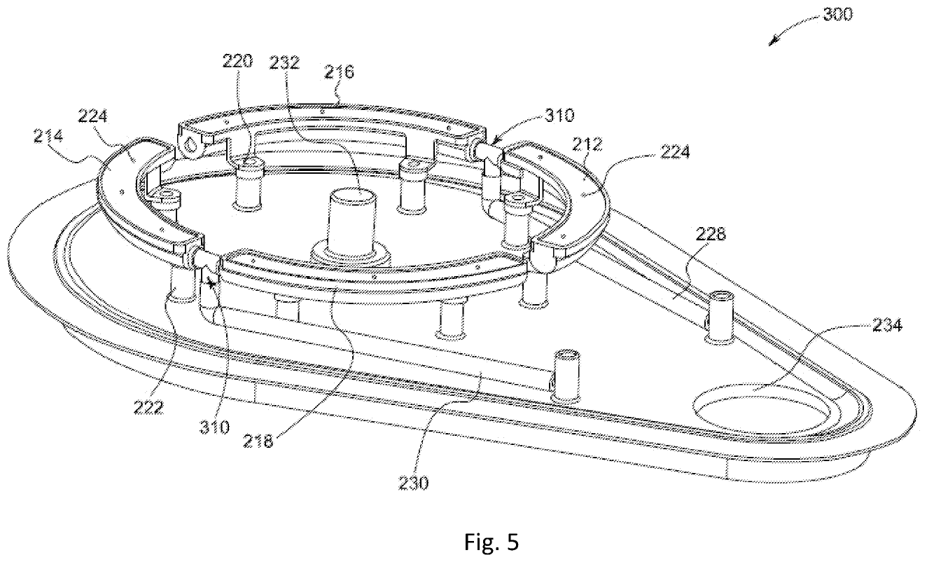

Referring now to FIG. 5, a sparger assembly 300 according to another embodiment of the invention is illustrated. The sparger assembly 300 is similar in configuration to the sparger assembly 200 of FIG. 4, where like reference numerals designating like parts. Rather than each aeration manifold having a hose barb connector for connection with a gas supply line, however, a T-fitting 310 is utilized to fluidly interconnect two adjacent aeration manifolds (e.g., aeration manifold 212 and aeration manifold 216, and aeration manifold 214 and aeration manifold 218), as well as connect the gas supply lines 228, 230, respectively, to the aeration manifolds. In one implementation, the fluidly interconnected aeration manifolds may each have gas outlet openings 224 of the same size. In another implementation, the first pair of interconnected aeration manifolds (e.g., aeration manifolds 212, 216) may have gas outlet openings 224 of a size that is different from a size of the gas outlet openings 224 of the second pair of interconnected aeration manifolds (e.g., aeration manifolds 214, 218). In yet another implementation, all of the manifolds may have gas outlet openings 224 of the same size.

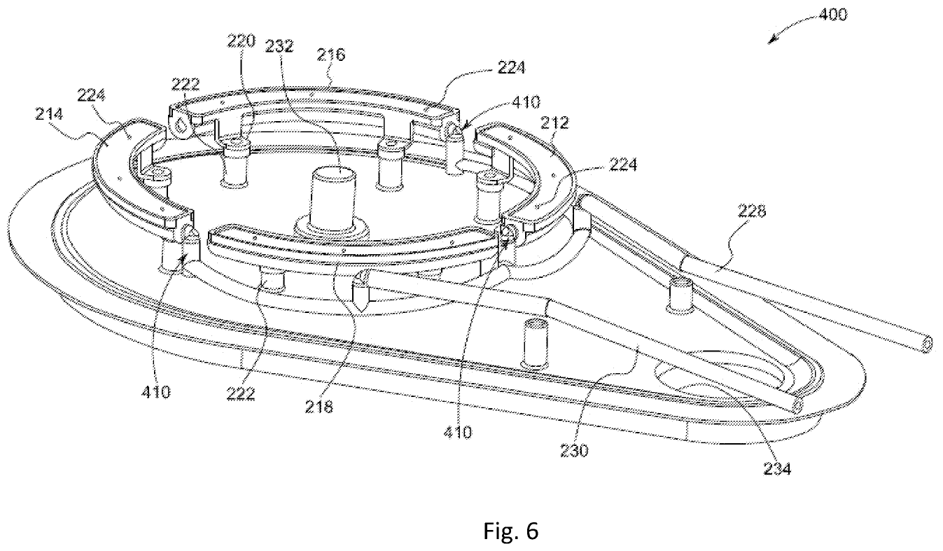

With reference to FIG. 6, a sparger assembly 400 according to another embodiment of the invention is illustrated. The sparger assembly 400, is similar in configuration to the sparger assembly 200 of FIG. 4, where like reference numerals designating like parts. Rather than each aeration manifold having a hose barb connector for connection with a gas supply line, however, an elbow fitting 410 is utilized to connect the gas supply lines 228, 230, respectively, to the aeration manifolds 212, 214, 216, 218. For example, elbow fittings 410 may be utilized to connect a first gas supply line 228 to the aeration manifolds 212, 216, as well as connect a second gas supply line 230 to the aeration manifolds 214, 218. As described above, some of the aeration manifolds may be configured with gas outlet openings 224 that have a different size than those of other aeration manifolds. In an embodiment, the aeration manifolds that are connected to a common supply line may have the same size gas outlet openings 224.

While FIGS. 3-6 illustrate sparger assemblies having two or four discrete aeration manifolds, it is contemplated that base plate may be manufactured with support posts 222 that are configured to receive three, or more than four, aeration manifolds of any partial-circle shape (i.e., any segment of a circle). In particular, the sparger assembly may include any number of arc-shaped aeration manifolds that together form a broken (or unbroken) circular arc. In an embodiment, the individual arc components may be isolated components in a generally circular or annular arc, which may be manufactured through additive manufacturing technologies. The base plate therefore allows the sparger assembly to be easily configured according to user preferences, and easily adapted to the particular bioprocess being carried out in the bioreactor system 210. As discussed above, the aeration manifolds may be configured for removable connection to the base plate to allow for easy customization of the sparger assembly.

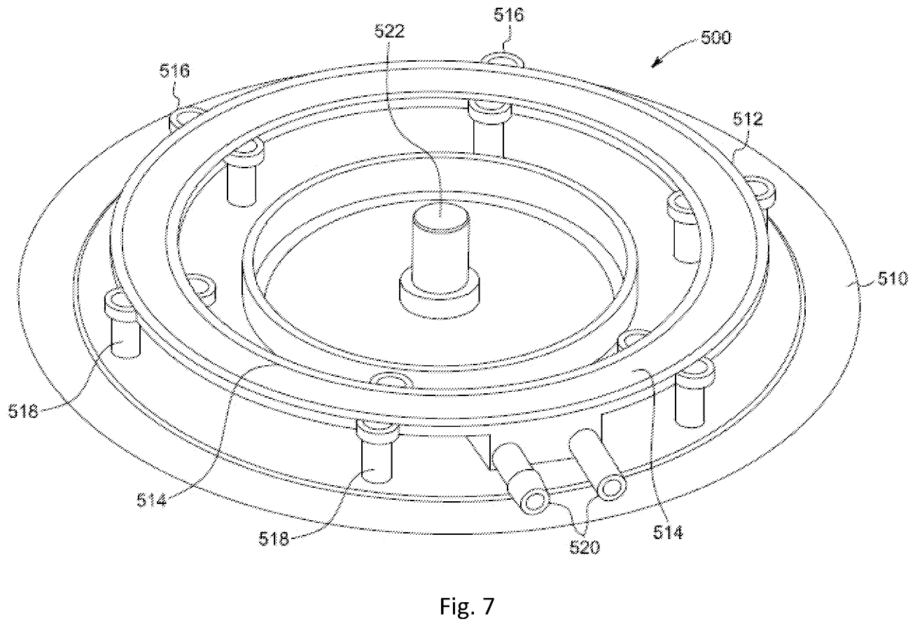

Turning now to FIG. 7, a sparger assembly 500 according to another embodiment of the invention is illustrated. As shown therein, the sparger assembly 500 includes a generally circular base plate 510 and an annular aeration manifold 512 removably connected to base plate 510. Similar to the embodiments discussed above, the aeration manifold 512 includes a plurality of gas outlet openings 514 and is raised above the base plate 510. In an embodiment, the aeration manifold 512 may include a plurality of feet 516 that are received by stand-offs or posts 518 of the base plate 510 to support the manifold 510 in vertically-spaced relation to the base plate. The aeration manifold 510 may also include one or more tubing connectors 520 for connecting one or more gas supply lines to the aeration manifold 510, in the manner described above. Similar to the embodiments described above, the base plate 510 may include a vertically-extending mounting shaft 522 located in the center of the aeration manifold 512 for receiving an impeller assembly.

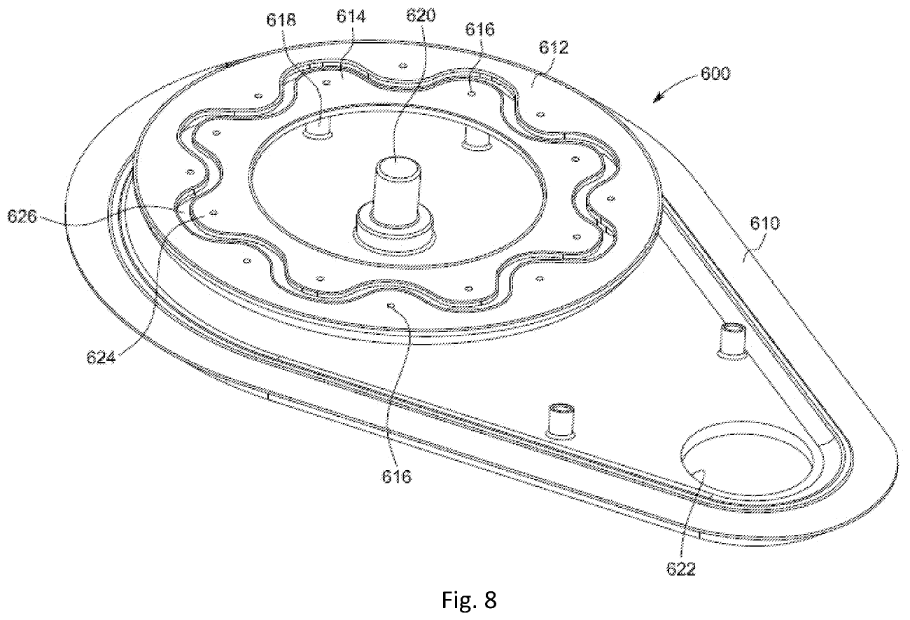

With reference to FIG. 8, a sparger assembly 600 according to yet another embodiment of the invention is shown. The sparger assembly 600 includes base plate 610 and a pair of nested aeration manifolds 612, 614 connected to base plate 610. Similar to the embodiments discussed above, each aeration manifold 612, 614 includes a plurality of gas outlet openings 616 and is raised above the base plate 610 (e.g., supported on projecting posts 618 that extend upwardly from the base plate 610. In an embodiment, the aeration manifolds 612, 614 are removably coupled to the base plate 610 and include tubing connectors (not shown) for connecting one or more gas supply lines (not shown) to the aeration manifolds 612, 614, in the manner described above. Similar to the embodiments described above, the base plate 610 may include a vertically-extending mounting shaft 620 located in the center of the aeration manifolds 612, 614 for receiving an impeller assembly of the bioreactor system 10. Moreover, the base plate 610 may include an aperture 622 or fitting for fluid coupling with drain tubing for draining or harvesting of the contents of the flexible bag 20.

As shown in FIG. 8, the aeration manifolds 612, 614 may have a pleated or sprocket shape. In particular, in an embodiment, the outer aeration manifold 612 may have an inner periphery that is generally sprocket shaped, and the inner aeration manifold 614 may have an outer periphery that is likewise generally sprocket shaped. The inner aeration manifold 614 may be sized and oriented so that the ‘teeth’ or peaks 624 of the inner aeration manifold 614 are received in corresponding recesses or grooves 626 in the outer aeration manifold 612. In an embodiment, the gas outlet openings 616 of the aeration manifolds 612, 614 may be the same or a different size.

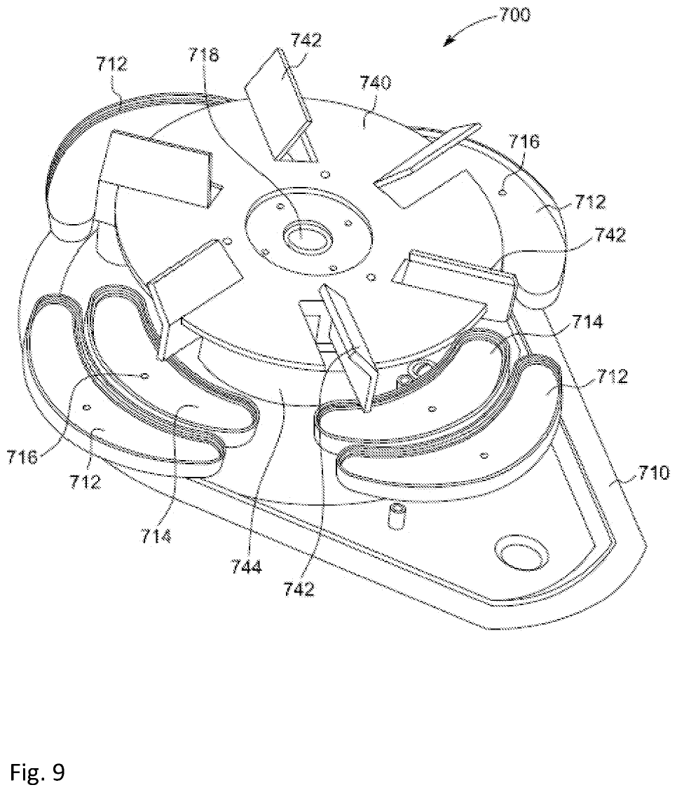

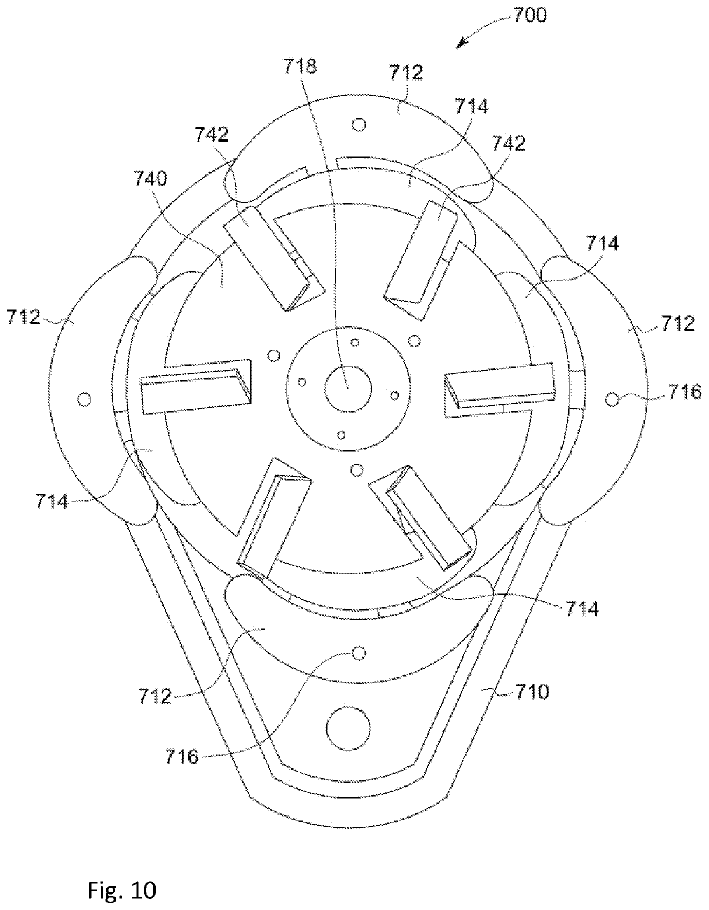

FIGS. 9 and 10 illustrate yet another embodiment of a sparger assembly 700 having a base plate 710 and plurality of aeration manifolds supported on the base plate in raised or spaced vertical relationship to the base plate 710, according to another embodiment of the invention. As shown therein, the aeration manifolds may include a plurality of outer, arch-shaped aeration manifolds 712 and a plurality of inner, arch-shaped manifolds 714 nested with, or positioned at a radial location inward of, the outer aeration manifolds 712. The aeration manifolds 712, 714 each include at least one gas outlet opening 716, the function of which has been hereinbefore described. The aeration manifolds 712, 716 are supported in a raised position above the base plate 710 by a plurality of posts or projections (not shown), as likewise hereinbefore described.

In an embodiment, the inner aeration manifolds 714 and outer aeration manifolds are raised above the support plate 710 substantially the same distance. In another embodiment, as best shown in FIG. 9, the inner aeration manifolds 714 are positioned closer to the top surface of the base plate 710 than are the outer aeration manifolds 712. In this respect, the outer aeration manifolds are raised above the base plate 710 to a greater extent than the inner aeration manifolds. This configuration allows the inner aeration manifolds 714 to be positioned beneath the blades of an impeller assembly 740 received on the impeller support shaft 718 (via a hub 744), and allows gas from the inner aeration manifolds 714 to be released through the gas outlet openings 716 thereof beneath the blades 742 of the impeller assembly 740. As shown in FIG. 10, gas from the outer aeration manifolds 712 may be released through the gas outlet openings 716 thereof at a radial location outward of the blades 742 of the impeller assembly 740, due to the positioning of the outer aeration manifolds 712 radially outward of the impeller blades.

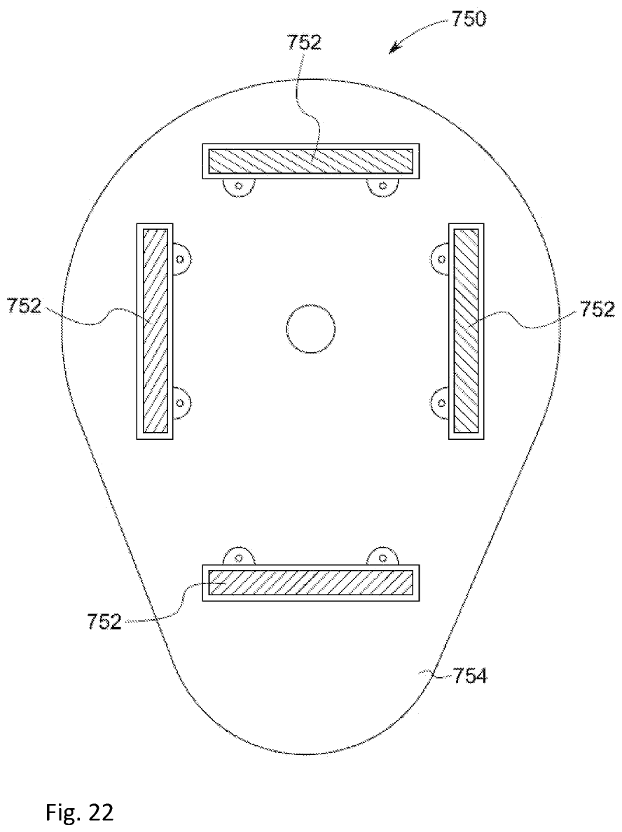

While the sparger assemblies of the invention have hereinbefore been described as having sparger elements/aeration manifolds that are arc or arch shaped, and arranged in a manner so as to form a circle or portion of an arc, the invention is not so limited in this regard. In particular, the aeration manifolds, themselves, may have any shape desired (e.g., rectangular, triangular, ovular, etc.) and may be arranged in an annular, circular, rectangular or any polygon shape. Other arrangements of the aeration manifolds on the base plate are also possible. For example, FIG. 22 illustrates a sparger assembly 750 having aeration manifolds 752 that are generally rectangular in shape, and which are removably mounted to the base plate 754 to form a generally rectangular array. In any embodiment, each aeration manifold may be separately or individually connected to a supply of a gas or gasses so that multiple gases can be delivered to each aeration manifold segment, as desired.

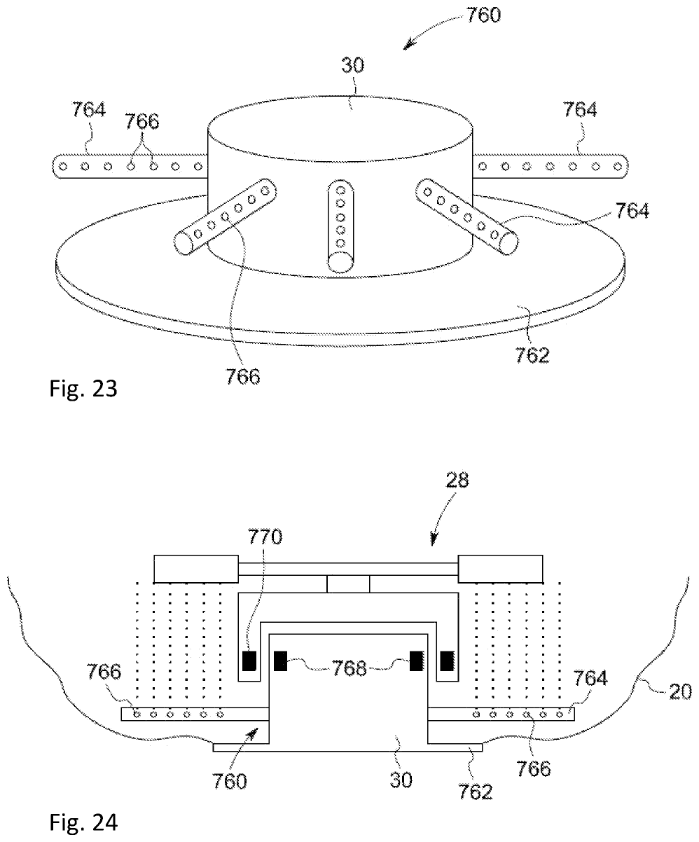

Turning to FIGS. 23 and 24, a sparger assembly 760 according to yet another embodiment of the invention is shown. Rather than having aeration manifolds that are mounted in vertically-spaced relation to the base plate, however, the sparger assembly 760 includes a base plate 762 having a hub (e.g., magnetic hub 30), and sparger elements or aeration manifolds 764 that extend radially from the hub 30. While not mounted to the planar portion of the base plate 762, the aeration manifolds are vertically spaced from the base plate. The aeration manifolds 764 have gas outlet openings 766, holes or pores that allow for the dispersion of gas into the interior of the flexible bioreactor bag 20, as described above. The aeration manifolds 764 may be removably coupled to the hub 30, although in some embodiments the aeration manifolds 764 may be permanently affixed to the hub 30. As shown in FIG. 24, and as discussed above, the magnetic hub 30 may include magnets 768 that cooperate with magnets 770 of the impeller 28 to rotationally drive the impeller 28.

In connection with the embodiments described above, by providing a sparger assembly that includes aeration manifolds for gas distribution that are raised from the base plate (or at least above a bottom surface of the vessel), sparge gas can be input into the bioreactor in close association with the impeller, which provides for more efficient gas dispersion in order to achieve a high gas surface area and bubble size distribution. Moreover, because the aeration manifolds are removably connected to the base plate, the sparger assembly may be universally configurable and adaptable to provide almost any gas distribution profile desired. In particular, the modular nature of the sparger assemblies described herein (i.e., base plate and removable aeration manifolds) allows for easy customization and creation of a sparger assembly, including customization of gas outlet height, gas outlet opening location, sparging ‘density’, etc.

In any of the embodiments described above, the interior of the aeration manifolds may be designed for optimized flow distribution such as, for example, by using a manifold groove system that promotes reduced pressure losses. In some embodiments, various components of the sparger assemblies, including the aeration manifolds, may be manufactured through additive manufacturing, which can be used to provide transitions from solid to porous materials with incorporated fluid channels to reduce the number of parts and ease of assembly. While the embodiments described above disclose hollow aeration manifolds having gas outlet openings, the manifolds may also be comprised of a porous frit wherein the openings for gas release are the pores in the porous frit.

In an embodiment, the pattern of apertures, holes or pores in the aeration manifolds of the spargers described herein can be any regular geometric pattern or a random pattern. In an embodiment, the apertures of one or more of the aeration manifolds may be arranged in a pattern which is configured such that the spacing between the apertures, holes or pores, s, is greater than the diameter of the gas bubbles that are produced by an aperture, hole or pore of diameter, d. Having a spacing between the apertures, holes or pores which is greater than the gas bubbles diameter assists in preventing adjacent gas bubbles from coalescing, as it keeps the bubbles from contacting each other at the surface of the sparger element/aeration manifold. The diameter of a gas bubble produced by an aperture, hole or pore of a specific diameter is dependent not only on the diameter of the hole or pore but is also greatly affected by factors such as the surface energy of the material from which the sparger is constructed and also on the physical and chemical properties of the liquid in which the bubbles are being created since that affects the surface tension of the air/liquid interface of the gas bubble surface.

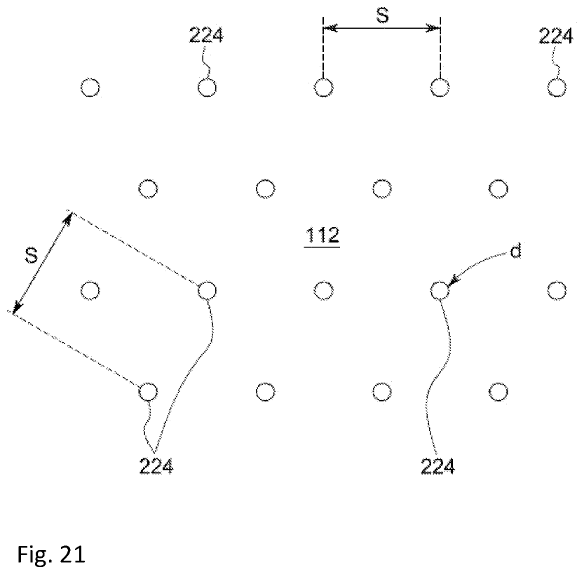

With reference to FIG. 21, an example of a geometric pattern for the location of the openings, holes or pores in the surface of an aeration manifold is shown. As illustrated in FIG. 21, the number of holes (e.g., holes 224) in the sparger element/aeration manifold (e.g., aeration manifold 112) is maximized by arranging the holes in a pattern of equilateral triangles, where the holes are located at the apexes of the triangles. This pattern is sometimes also referred to as a hexagonal pattern. This pattern maximizes the number of holes that could be created in a sparger element with a specific surface area. In the equilateral triangle pattern of FIG. 21, all openings, holes or pores are at equal distances from adjacent openings, holes or pores. Other geometrical patterns such as a simple rectangular grid could also be used, without departing from the broader aspects of the invention. When holes or pores are located at the corners of a rectangular grid, then adjacent holes in the sparger element are located at two different distances, the desired horizontal and vertical distance and the longer distances on the diagonals. Thus, for a specific desired minimum spacing between adjacent holes, the spacing of the holes on the diagonals would be at a distance which is greater than the desired minimum distance. Such a rectangular pattern would result in a smaller number of holes in a sparger element of a specific surface area than would be the case for the more efficient equilateral triangle pattern.

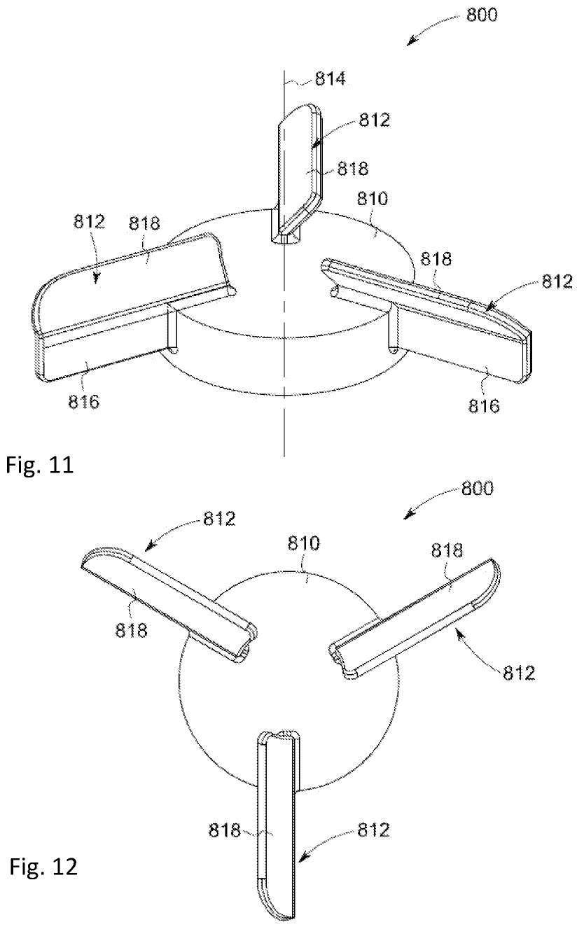

Referring now to FIGS. 11-18, various configurations of the impeller assembly of the bioreactor/bioprocessing system 10 are shown. With specific reference to FIGS. 11-14, in one embodiment, an impeller assembly 800 includes a hub 810 and at least one blade 812 extending radially from the hub 810. The hub 810 is rotatable about a vertical axis 814 that extends through the center of the hub 810. In an embodiment, the hub 810 may be a magnetic hub configured to be driven by the magnetic drive system or motor (e.g., motor 34 of FIG. 2) positioned exterior to the flexible bag 20 and vessel 12.

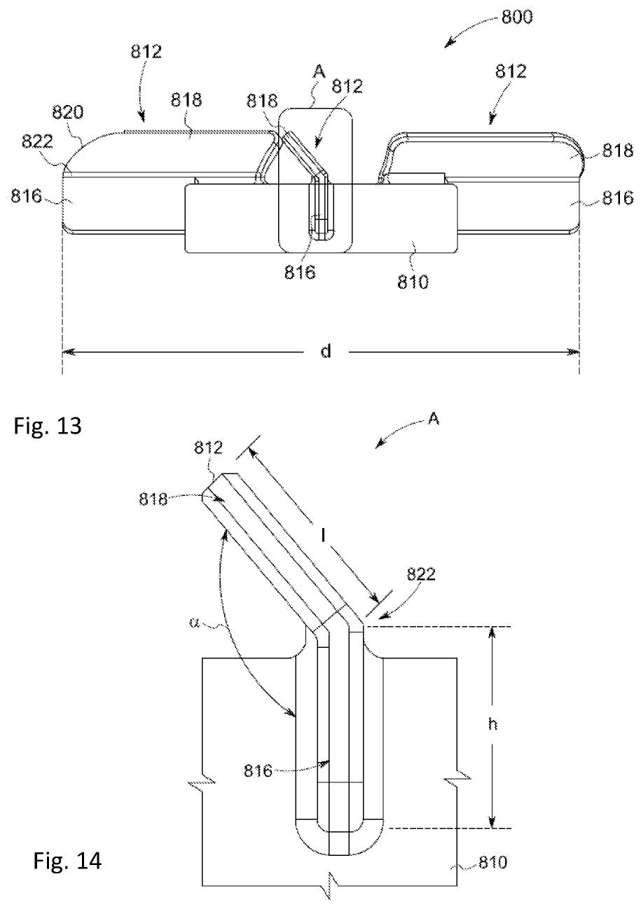

While the impeller assembly 800 is shown in FIGS. 11-14 as having three blades 812, the impeller assembly 800 may have fewer than three blades (e.g., one blade or two blades) or more than three blades, without departing from the broader aspects of the invention. The blades 812 may be equally spaced from one another about the hub 810. For example, where the impeller assembly 800 has three blades 812, the blades 812 may be spaced 120° apart. The blades 812 each include a first, substantially vertical portion 816 and a second, non-vertical, non-horizontal, angled portion 818 which extends upwardly from the first portion 816. The first portion 816 and the second portion 818 are shown as being substantially planar, although in some embodiments, it is contemplated that one or both of the first and section portions 816, 818 of the blades 812 may have a curved or arcuate shape. As best shown in FIG. 13, the second, angled portion 818 includes a radiused portion 820 at a distal end of the blade 812. A radius 822 is also formed at the intersection between the first, vertical portion 816 and the second, angled portion 818.

With specific reference to FIGS. 13 and 14, the impeller assembly 800 has a diameter, d, defined as the longest linear dimension from blade tip to blade tip. In an embodiment, the diameter, d, of the impeller may be in a range from about ¼ to about ½ the inner diameter of the vessel 12. As best shown therein, the first, vertical portion 816 and the second, angled portion form an angle, a, therebetween. In an embodiment, the angle, a, is between about 100 degrees to about 180 degrees. In an embodiment, the angle, α is about 135 degrees, such that the second, angled portion extends at an upward angle of about 45 degrees from horizontal.

As alluded to above, the impeller assembly 800 may be seated on the bottom of the flexible bag 20 in close association with a sparger assembly. For example, the impeller assembly 800 may be connected to a base plate of one of the sparger assemblies disclosed herein, such that the impeller blades 812 are in close association with the gas outlet openings of the sparger assembly. Through testing, it has been shown that the vertically straight portion 816 of the blades 812 of the impeller assembly 800 is particularly efficient in breaking the bubbles input into the flexible bag 20 by the sparger assembly, and delivers high power to the bioreactor system 10. In addition, testing has demonstrated that the angled portion 818 of the blades 812 facilitates mixing of the contents of the flexible bag 20. Accordingly, this combination of straight and angled blade portions yields improved bubble break-up and efficient gas distribution (kLa) with optimum power consumption (i.e., without requiring greater power input or agitation at very high speeds, which can cause shear damage and produce eddies that are harmful to the cells).

In this respect, the impeller assembly 800 optimizes bulk mixing and efficient gas distribution at the gas sparger to provide high oxygen transfer rates and kLa values, which is desirable in intensified cell culture and/or microbial applications. In contrast to existing systems and devices, the impeller assembly 800 achieves this performance while maintaining a relatively low profile (i.e., it remains bottom driven and sits closely to the bottom of the bag 20, allowing for the bag to still be easily collapsed for storage and transport). This simple design also allows for easy user installation and configuration. In particular, in some embodiments, the impeller assembly 800 may be quickly and easily positioned on the mounting shaft of the base plate of a sparger assembly, in the manner hereinbefore described.

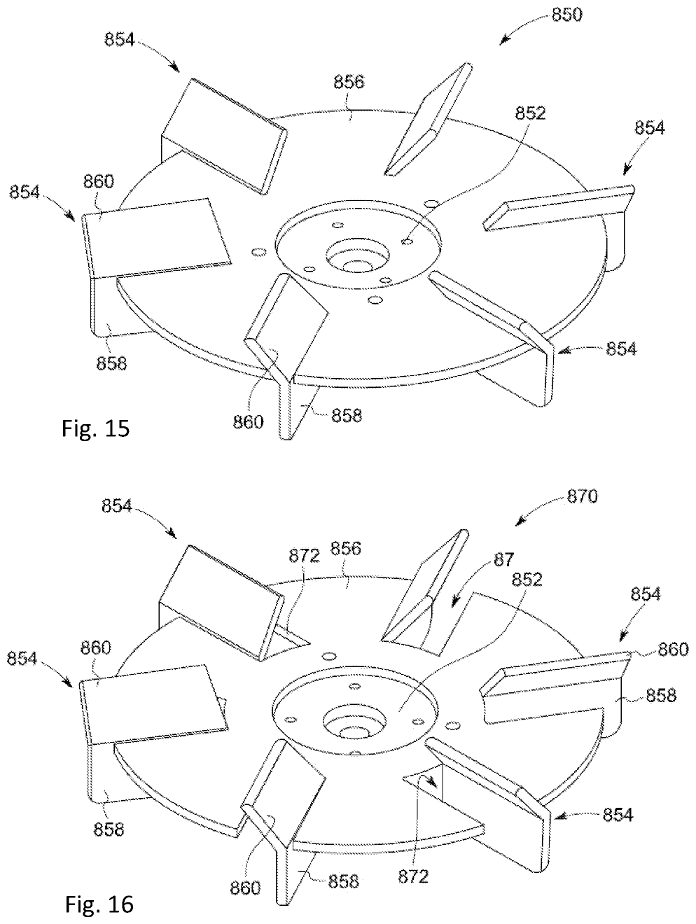

Referring now to FIG. 15, an impeller assembly 850 according to another embodiment of the invention is illustrated. As shown therein, the impeller assembly 850 includes a hub 852 and at least one blade 854 connected to the hub 852. Like the embodiment of FIGS. 11-14, the hub 852 is rotatable about a vertical axis that extends through the center of the hub 852. In an embodiment, the hub 852 may be a magnetic hub configured to be driven by the magnetic drive system or motor (e.g., motor 34 of FIG. 2) positioned exterior to the flexible bag 20 and vessel 12. In an embodiment, the hub 852 may be formed as (or be otherwise integrated with) a generally flat disc 856 to which the blades 854 extend.

The blades 854 are substantially similar to the blades 812 of the impeller assembly 800 of FIGS. 11-14, and each include a first, substantially vertical portion 858 and a second, non-vertical, non-horizontal, angled portion 860 which extends upwardly from the first portion 816. The first portion 858 and the second portion 860 are shown as being substantially planar, although in some embodiments, it is contemplated that one or both of the first and section portions 858, 860 of the blades 854 may have a curved or arcuate shape. As shown in FIG. 15, in an embodiment, the first, vertical portion 858 extends downwardly from the distribution disc 856, while the second portion 860 extends upwardly at an angle from the distribution disc 856. The blades 854 may terminate at the outer periphery of the distribution disc 856, or may extend beyond such outer periphery to an extent, as shown in FIG. 15.

Turning now to FIG. 16, another impeller assembly 870 according to an embodiment of the invention is illustrated. The impeller assembly 870 is substantially similar to the impeller assembly 850 of FIG. 15, where like reference numerals designate like parts. As shown in FIG. 16, however, the distribution disc 856 may additional include radial slots 872 adjacent to each (or at least some of) the blades 854.

While the impeller assemblies 850, 870 of FIGS. 15 and 16 have six blades, the impeller assemblies may have more or fewer than six blades, without departing from the broader aspects of the invention. In the embodiments of FIGS. 15 and 16, the vertical blade portions of the blades provide for efficient radial liquid flow, while the angled blade portions allow for axial fluid flow. Moreover, the distribution disc 856 functions to entrap and enrich air/gas bubbles from the sparger assembly before dispersing them. The slots 872 in the distribution disc 856, as shown in FIG. 16, allow for a different bubble distribution pattern. These impeller assembly designs provide for proper mixing and mass transfer of oxygen from the gas phase to the liquid phase, which is essential for cell culture for biopharmaceutical manufacturing, for example, where at very high cell concentrations the demand for oxygen and uniform mixing is very high. In addition, the impeller assemblies shown herein effectively disperse the gas bubbles from the sparger and mix efficiently, without agitating at very high speeds, which can cause shear damage and produce harmful eddies.

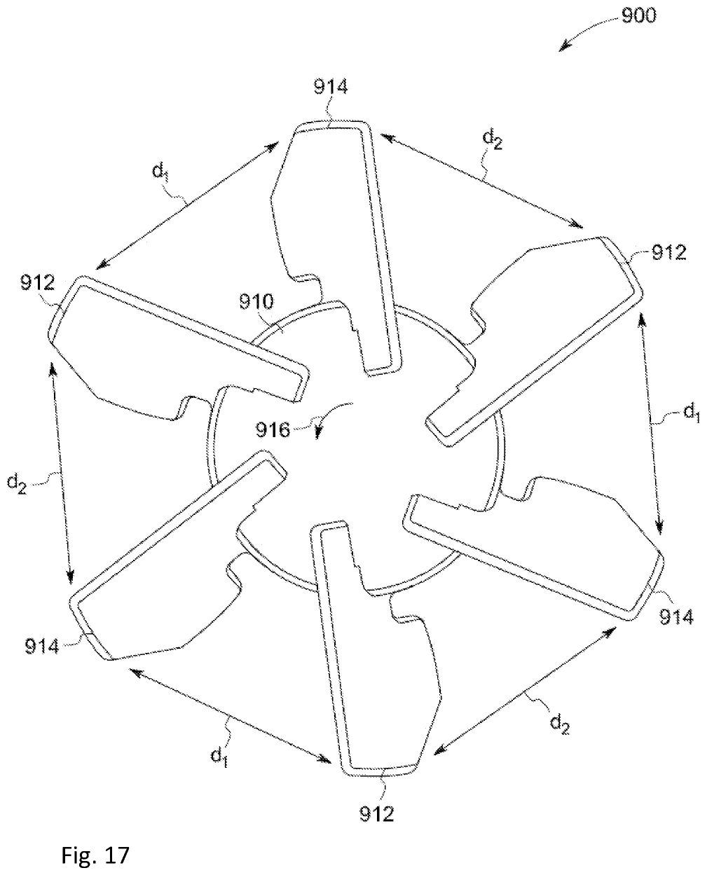

Turning now to FIG. 17, an impeller assembly 900 according to another embodiment of the invention is illustrated. The impeller assembly 900 includes a hub 910 and a plurality of blades 912, 914 attached to the hub and extending radially outward from the hub 910.

In an embodiment, the hub 910 is a magnetic hub configured to be driven by an external magnetic drive system or motor, as discussed above. While FIG. 17 illustrates impeller assembly 900 having six blades 912, 914, the impeller assembly may have fewer or more than six blades without departing from the broader aspects of the invention.

In an embodiment, one or more the blades 912, 914 are connected to the hub 910 at angles offset from a radial line extending from the impeller axis. For example, blades 912 may be angled forward of a radial line extending from the impeller axis with respect to a direction of rotation 916 of the impeller assembly 900, while blades 914 may be angled rearward of a radial line extending from the impeller axis with respect to the direction of rotation 916 of the impeller assembly 900. As shown in FIG. 17, the blades may alternate between being forward-angled or rearward-angled. In such an implementation, this blade configuration results in longer and shorter distances between the tips of the blades as compared to uniform distances between the blade tips in the absence of such angled or canted blades. For example, a distance, d1, between the tip of a rearwardly-angled blade 914 and the next adjacent forwardly-angled blade 912 (moving in the direction of rotation of the impeller assembly 900) is increased as compared to the distance between blade tips if the blades were oriented along a radial line extending from the center of the hub 910. In addition, a distance, d2, between the tip of a forwardly-angled blade 912 and the next adjacent rearwardly-angled blade 914 (moving in the direction of rotation of the impeller assembly 900) is decreased as compared to a distance between blade tips if the blades were oriented along a radial line extending from the center of the hub 910. In this respect, the impeller assembly 900 has alternating longer and shorter distances between the tips of the blades.

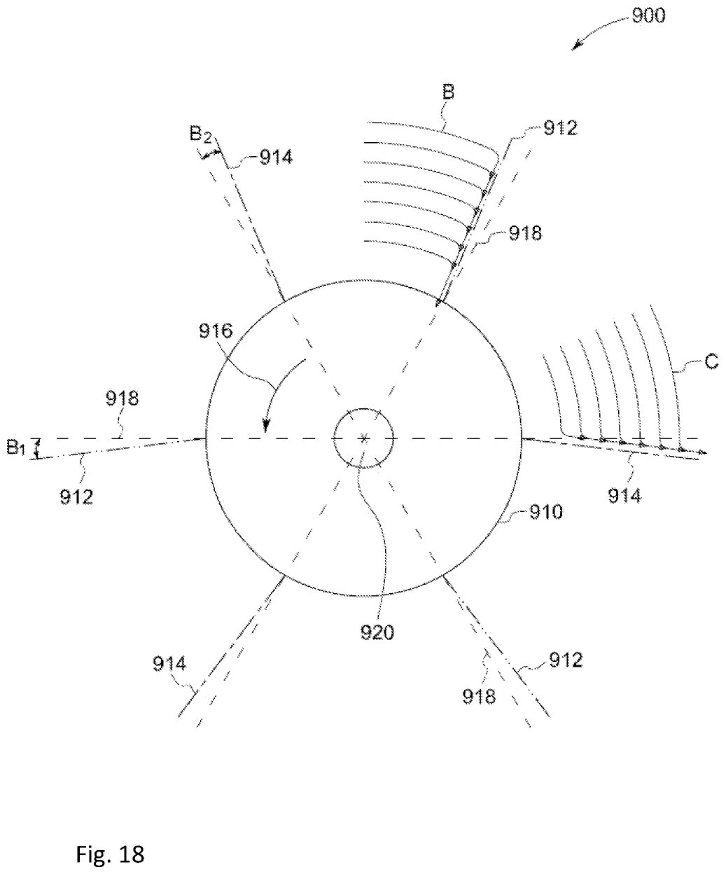

This canted configuration of the blades 912, 914 of the impeller assembly is more clearly shown in FIG. 18. As illustrated therein, alternating blades 912 are oriented at a leading angle, β1, with respect to true radial lines 918 extending from the central axis 920 of the impeller assembly 900. In contrast, alternating blades 914 are oriented at a lagging angle, β2, with respect to true radial lines 918 extending from the central axis 920 of the impeller assembly 900. In an embodiment, the leading angle β1 of the blades 912 may be equivalent to the lagging angle, β2, of the blades 914. For example, in an embodiment, leading angle β1 and lagging angle β2 may be between about 5 degrees and about 30 degrees. In another embodiment, leading angle β1 and lagging angle β2 may be between about 5 degrees and about 10 degrees. In yet another embodiment, leading angle β1 and lagging angle β2 may be about 7 degrees. In other embodiments, the leading angle, β1, of the blades 912 may be different from the lagging angle, β2, of the blades 914. In yet other embodiments, one or more of the blades 912 may having a different leading angle, β1, than at least another of the blades 912. Similarly, one or more of the blades 914 may having a different lagging angle, β2, that at least another of the blades 914. It is contemplated that the number of blades with leading angles may be the same or different than the number of blades with lagging angles.

In operation, the blades 912 oriented at leading angles with respect to a true radial line 918 extending from the central axis 920 function to pull liquid inwardly towards the hub 910, in the direction of arrows B, as shown in FIG. 18. Conversely, the blades 914 oriented at lagging angles with respect to a true radial line 918 extending from the central axis 920 function to push liquid away from the hub 910, in the direction of arrows C, as shown in FIG. 18. Accordingly, the impeller assembly 900 can be utilized to increase mixing effectiveness, which can improve oxygen transfer within the bioreactor system 10. It is contemplated that the blade orientation/canting aspects of the invention may be employed in conjunction with existing blade geometries/shapes/configurations known in the art to improve impeller mixing capability.

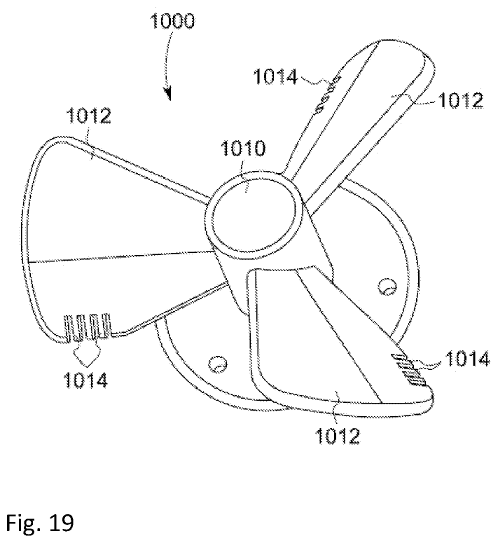

With reference to FIG. 19, an impeller assembly according to another embodiment of the invention is illustrated. The impeller assembly 1000 includes a hub 1010 and a plurality of blades 1012 mounted to the hub 1010. While the impeller assembly 1000 of FIG. 19 has three blades 1012, fewer or more than three blades may be employed without departing from the broader aspects of the invention. In an embodiment, the impeller assembly 1000 is a marine-type impeller having arcuate or curved blades 1012. As illustrated in FIG. 19, in an embodiment, one or more of the blades 1012 includes a plurality of slots 1014. In an embodiment, the slots 1014 are generally vertically-extending slots and are positioned at a location on the blades 1012 that is generally aligned with, in a vertical direction, the location on a sparger assembly where the sparge gas is released into the flexible bag 20. In an embodiment, the slots 1014 are formed on a forward or leading edge of the blades 1012.

In use, the impeller assembly 1000 may be mounted to the mounting shaft of a sparger assembly, as discussed above. As indicated above, the slots 1014 are positioned so that when the blades 1012 rotate, the slots 1014 pass closely over the gas outlet openings in the sparger assembly.

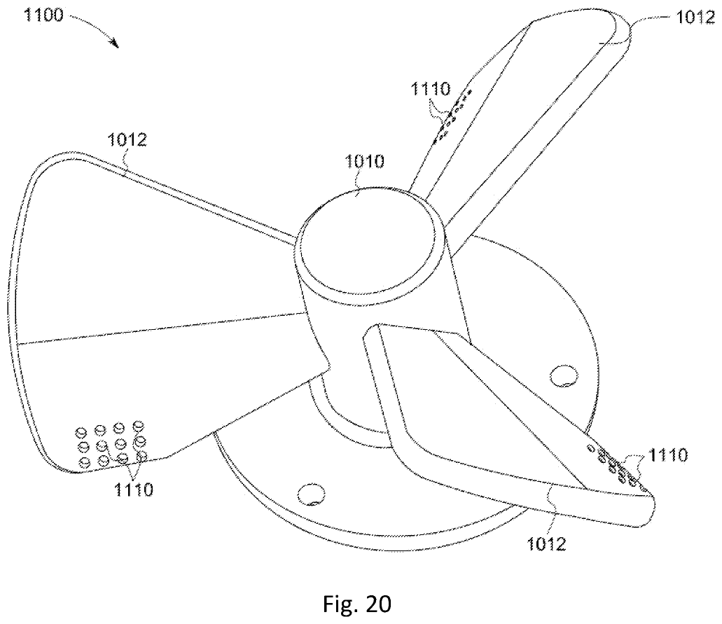

Referring to FIG. 20, a similar impeller assembly 1100 is illustrated. Rather than having slots in the forward edge of the blades 1012, however, an array of depressions, holes or apertures 1110 may be formed in a leading edge of the blades 1012. Similar to the embodiment of FIG. 19, the apertures 1110 are disposed at a location that generally corresponds to the location of the gas outlet openings of the sparger assembly on which the impeller assembly 1100 is disposed.

It is contemplated that slots or apertures may be integrated with any existing impeller designs or configurations for a bioreactor system, as well as the impeller assembly configurations described herein. By utilizing an impeller with slots or apertures in the area of the blade that passes closely over the gas outlet openings of the sparger assembly, the interfacial contact between the blades of the impeller and the fluid within the flexible bag 20 may be increased. Accordingly, the impeller assemblies 1000, 1100 provide for more efficient gas distribution at the gas sparger to provide high oxygen transfer rates and kLa values desired for enhanced cell culturing, without increasing the power requirements on the impeller drive system.

Embodiments of the impeller assemblies and sparger assemblies disclosed herein provide various means of increasing kLa of a bioreactor system (i.e., achieving more efficient gas distribution) to support intensified cell culture and/or microbial applications. It is contemplated that the impeller assemblies disclosed herein may be utilized in conjunction with any existing sparger assembly. Similarly, the sparger assemblies disclosed herein may be utilized in connection with a number of existing impeller assemblies. Still further, it is envisioned that any of the impeller assemblies disclosed herein may be utilized in conjunction with any of the sparger assemblies also disclosed herein, to provide both improved bulk mixing and efficient gas dispersion. In this respect, the configuration of both the impeller assemblies and sparger assemblies of the invention facilitates simple user manipulation or configuration of a combined impeller and sparger assembly. In particular, the impeller and/or sparger assemblies of the invention can be easily manipulated (e.g., by interchanging the aeration manifolds on the sparger and/or connecting different impellers to the sparger base plate) to achieve almost any level of bulk mixing or gas dispersion desired, depending on the particular cell culturing or bioprocessing operations being carried out within the bioprocessing system 10.

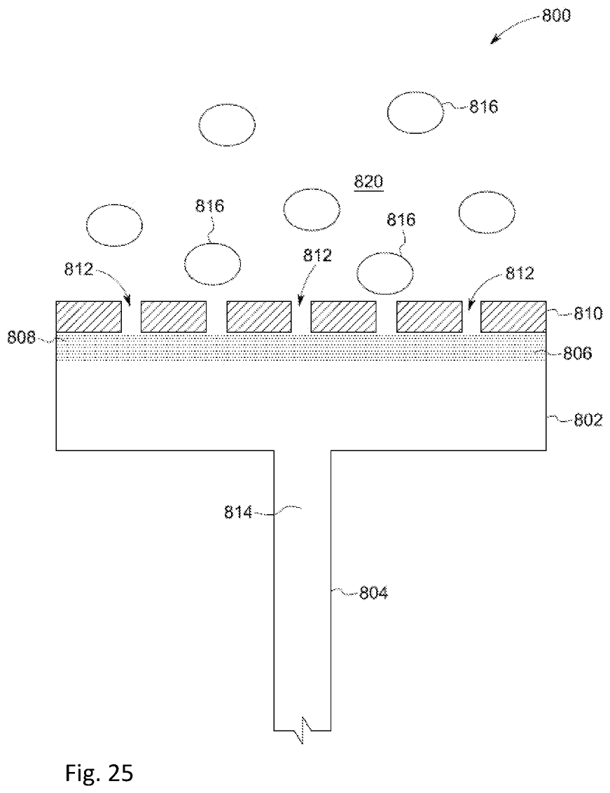

In addition to the impeller and sparger assemblies described above, the present invention further provides various sparger assembly constructions that provide for additional operational advantages over existing sparer devices. For example, FIG. 25 illustrates a sparger assembly 800 that provides sparge gas to a bioreactor vessel in a manner similar to existing drilled hole spargers, but which advantageously inhibits or prevents backflow of liquid from the bioreactor vessel into the sparge gas supply line, including at low or zero flow rates.

As shown in FIG. 25, the sparger assembly 800 includes a housing having one or more tube connectors 804 forming an inlet configured for mating connection with a gas supply line (not shown), enabling sparge gas from a supply to be delivered to the housing 802. In an embodiment, the tube connector 802 is a hose barb connector, although other connector types known in the art may also be utilized without departing from the broader aspects of the invention. The housing 802 may take any shape generally known in the art, such as circular. The sparger assembly 800 further includes a first layer or section 806 having a plurality of pores 808 of a first size and mounted within the housing 802, and a second layer or section 810 mounted within the housing and disposed above the first layer 806 and forming a sandwich therewith, and having a plurality of pores, apertures or openings 812 of a second size. The size of the openings 812 in the second layer 810 is greater than the size of the openings or pores 808 in the first layer 806, as described hereinafter,

In an embodiment, the first layer or section 806 is formed from a hydrophobic material and may be, for example, a sintered part such as a porous frit. In an embodiment, the first layer 806 may be formed from a variety of hydrophobic materials such as a polymer material including, for example, polyethylene, polytetrafluoroethylene (PTFE), polypropylene, fluorocarbons, etc., and the pores may be formed therein via any means known in the art. Regardless of the specific material and manufacturing method utilized, the size of the pores 808 of the first layer 806 is such that the first layer 806 is water impermeable and gas permeable, meaning that water is prevented from passing through the first layer 806, but gas (e.g., oxygen or carbon dioxide) is permitted to pass through the first layer 806 via the pores 808. In an embodiment, the size of the pores 808 in the first layer 806 is between about 2 and about 20 micrometers. In an embodiment, the first layer 806 is formed from a hydrophobic material and has a three-dimensionally interconnected pore structure such that gas is permitted to pass through the interconnected pores linearly and/or in a zig-zag or traversing manner so as to enter from the bottom of the first layer 806 and exit the top of the first layer 806.

The second layer or section 810, as described above, has openings 812 of a size that permit the passage of gas (and which may, in some embodiments, likewise permit the passage water therethrough). In an embodiment, the second section 810 may take the form of a drilled hole, marco sparger, having a plurality of discrete holes through the second section 810 that are monodisperse in size/diameter. In an embodiment, the size of the openings 812 in the second layer is between about 100 and about 500 micrometers in diameter.

In operation, the sparger assembly 800 is placed inside a bioreactor vessel, for example, inside flexible bag 20 of the bioreactor/bioprocessing system 10 in a manner heretofore known in the art. Various bioprocessing or cell culturing operations may then be carried out within the flexible bag 20, as known in the art. Sparge gas 814 is supplied to the housing 802 through connector 804, and passes through the porous first layer 806, and subsequently through the openings 812 in the second layer 810, where bubbles 816 of a desired size are formed and dispersed into the liquid within the interior 820 of the flexible bag 20. The size of the openings 812 in the second layer 810, and the flow rate of the gas, are chosen so as to produce bubbles 816 of a desired size (such as, for example, to provide a nominal gas flow through the liquid in the interior 820 of the bag to control the partial pressure of carbon dioxide). Typically, the size of the openings in such drilled hole, macro spargers is such that liquid from the interior of the bag may leak beyond the sparger and into the gas supply line, particularly at low gas flow rates or when sparging is ceased. The presence of the hydrophobic first layer 806 beneath the second layer or section 810, however, prevents the liquid from leaking past the first layer 806 (while simultaneously permitting the passage of spage gas from the housing 802 into the interior 820 of the bag during sparging).

The sparger 800 of the invention therefore allows for the formation of bubbles of a desired size typical of existing drill hole, macro spargers, but which also inhibits or prevents liquid backflow from the processing volume into the sparge gas supply or delivery line. It is contemplated that such sandwiched-layer sparger construction may be incorporated into any of the sparger configurations described herein (and as shown in, for example, FIGS. 3-10).

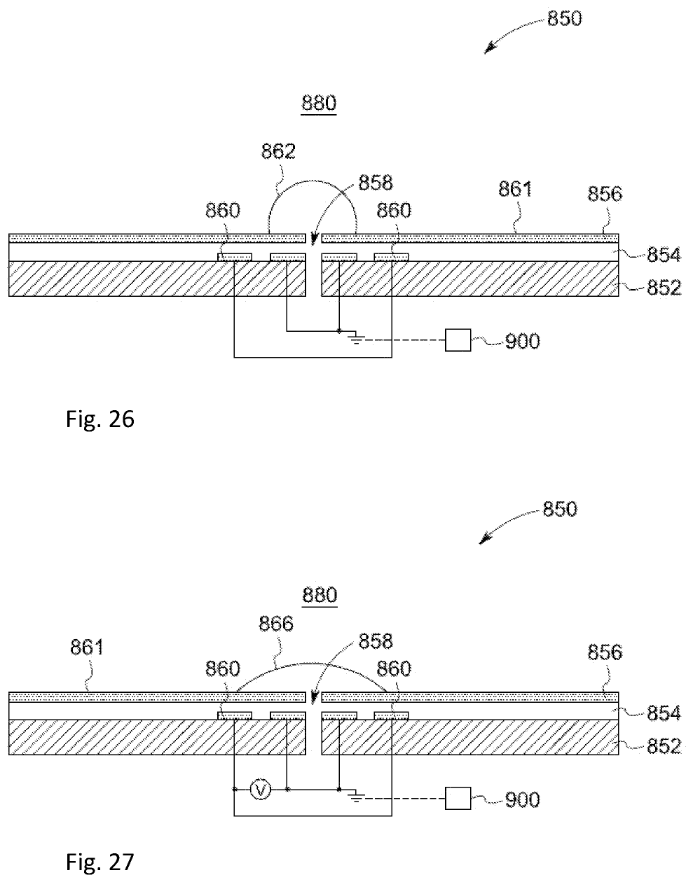

Turning now to FIGS. 26 and 27, a sparger assembly 850 according to another embodiment of the invention is illustrated. The sparger assembly 850 enables sparge gas bubble diameter and/or distribution to be selectively adjusted during the cell culturing process, as described hereinafter. As shown therein, the sparger assembly 850 includes a base layer 852, a dielectric layer 854 disposed above the base layer, and a top layer 856 disposed above the dielectric layer 854. In an embodiment, the top layer 856 is formed from a hydrophobic material. At least one opening 858 extends through at least the top layer 856 and is in fluid communication with a supply of sparge gas for delivering the sparge gas to the interior 880 of a bioprocessing vessel (e.g., flexible bag 20). As illustrated in FIG. 26, the opening 858 may extend through each of the layers (e.g., from the bottom surface of the base layer 852, to the upper surface 861 of the hydrophobic layer 856). While FIGS. 26 and 27 illustrate a single opening 858, the sparger assembly 850 may include a plurality of openings arrayed throughout the surface area of the sparger assembly. In an embodiment, the opening(s) 858 may be between about 50 micrometers to about 3 millimeters in diameter (depending on the particular application/objective such as oxygen transfer or carbon dioxide clearance).

In an embodiment, the layers 852, 854, 856 may be sandwiched together and mounted or received within a sparger housing (not shown) having a tube connector for selective connection to the supply of sparge gas. For example, the housing and tube connector may be constructed similar to those shown in the sparger assembly of FIG. 25.

As further shown in FIG. 26, the sparger assembly 850 further includes a plurality of electrodes 860 in contact with the dielectric layer 854 in close association with, or surrounding, the opening(s) 858. In an embodiment, the electrodes 860 are sandwiched between the base layer 852 and the dielectric layer 854. The electrodes 860 are in electrical communication with a voltage source for energizing the electrodes, as discussed hereinafter

It is envisioned that the electrodes 860 could take several forms and employ different shapes. For example, in one embodiment, the electrodes 860 may be arrayed in two or more concentric rings located around the openings 858. In an embodiment, there may be a concentric ring closely surrounding each opening with a larger common plane surrounding all of the concentric rings on the surface of the sparger. The rings which surround the openings could have a more complex shape, such as concentric rings with interdigitated fingers extending between the rings. In an embodiment, the electrode to electrode spacing could be as small as, for example, 50 um.

In some embodiments, the electrode 860 closest to the opening 858 may be in contact with the opening or within 50um of the opening. The electrode closest to the opening could be in contact with the opening such that it is part of the opening and take the form of a plated through-hole.

In an embodiment, the base layer 852 may be formed from, for example, a polyimide (Kapton), glass epoxy and/or ceramic materials commonly used in the electronics industry such as silicone dioxide (SiO₂). Any base layer material with questionable/unverified biocompatibility could be encapsulated/coated in other biocompatible materials such as polydimethylsiloxane (PDMS), Ethylene tetrafluoroethylene (ETFE), Polyvinylidene chloride (PVDC) in any area where product contact could occur. The electrodes in contact with the fluid inside the bioreactor bag could be gold plated which would make them biocompatible. In embodiments where the base layer is polyimide or glass epoxy, then printed circuit board fabrication techniques could be used to construct the sparger assembly 850 (e.g., spin coating followed by oven drying could be used to overlay the hydrophobic layer over the other two layers).

In operation, the sparger assembly 850 is placed inside a bioreactor vessel, for example, inside flexible bag 20 of the bioreactor/bioprocessing system 10 in a manner heretofore known in the art. Various bioprocessing or cell culturing operations may then be carried out within the flexible bag 20, as known in the art. Sparge gas, when supplied to the housing (not shown), passes through the openings 858 in the sparger assembly 850, initially forming bubbles, e.g. bubble 862, on the exposed upper surface 861 of the top layer 856. The bubbles 858 are subsequently released form the upper surface 861 of the top layer 856 and enter the liquid within the bioreactor vessel.
The contact angle the bubble 862 makes with the upper surface 861 of the top layer 856 is one of the governing factors that control how the bubble forms and thus how big it grows to before it detaches from the upper surface 861. In an embodiment, a voltage is applied to the electrodes 860, which varies the contact angle between the bubble(s) 862 and the upper surface 861. Therefore, applying a voltage to the electrodes 860 surrounding the opening(s) 858 allows for the diameter/size of the bubbles to be varied. This is illustrated in FIG. 27, whereby energizing the electrodes 860 by applying a voltage, V, changes the contact angle of the bubble with the upper surface 861 of the top layer 856. As a result, a bubble 866 having a greater diameter is formed.

The sparger assembly 850 of the invention therefore allows for bubble diameter to be selectively varied by altering the voltage applied to the electrodes, irrespective of the diameter of the openings(s) 858 (i.e., even with a fixed opening diameter). This allows the sparge gas bubble diameter distribution produced by the sparger assembly 850 to be continuously adjusted, as needed, to match, for example, the oxygen mass transfer requirements and/or the carbon dioxide clearance requirements during a bioreactor run.

The principle of operation of the sparger assembly 850 is similar to electrowetting on dielectric (EWOD) techniques currently used to manipulate fluids in microfluidic devices. Currently, spargers used in many bioprocessing fields, including in the pharmaceutical industry use fixed diameter, circular, pores/holes in the sparger element. It is contemplated, however, that the EWOD techniques described herein may be utilized in conjunction with different pore/opening geometries (such as a hole with a lobed cross section) and complementary electrode geometries/patterns to enable even greater or more precise control of bubble diameter. [000110] In an embodiment, the sparger assembly 850 may include various arrays of electrodes associated with a discrete set of (or single) sparge gas pore/opening. The arrays of electrodes may each be individually controllable by a controller 900 or microprocessor, i.e., each array of electrodes may be dynamically addressed electronically. This allows individual pores/openings or groups of pores/openings to be differentially activated, allowing for sections or patterns of individual pores/openings on a single sparger element or assembly to be adjusted to produce different distributions of bubble diameters, as desired.

The ability to control bubble diameter across the entire sparger assembly, and even vary bubble diameter at certain discrete areas across the sparger assembly relative to other discrete areas of the sparger assembly, provides a wider range of operational effectiveness than has heretofore been possible with existing devices and, particularly, sparger devices having fixed hole/opening sizes. The ability to selectively change the bubble diameter of the sparge gas allows for the generation of bubbles having a diameter that is, for example, efficient at the beginning of a cell culture process and does not cause excessive foaming when the cell density is low, and for selective adjustment mid-process, in real time, to produce bubbles having a different diameter that are more efficient when the cell density is higher. In some embodiment, the bubble diameter may be adjusted in the manner described herein in conjunction with varying the impeller speed to provide better and more efficient mass transfer control during the bioprocessing operation.

It is contemplated that the sparger construction shown in FIGS. 26 and 27 may be incorporated into any of the sparger configurations described herein (and as shown in, for example, FIGS. 3-10).

In an embodiment, a sparger assembly for a bioprocessing system is provided. The sparger assembly includes a first layer having a plurality of pores of a first size, and a second layer disposed above the first layer and having a plurality of holes of a second size, the second size being greater than the first size. The pores of the first layer and the holes of the second layer allow for the passage of a sparge gas through the first layer and the second layer. In an embodiment, the first size is sufficient to allow for passage of gas through the first layer, and to inhibit the passage of water through the first layer. In an embodiment, the first layer is formed form a sintered hydrophobic material. In an embodiment, the second layer is configured as a drilled hole sparging element. In an embodiment, the first size is between about 2 and about 20 micrometers. In an embodiment, the second size is between about 100 and about 500 micrometers. In an embodiment, the sparger assembly also includes a housing receiving the first layer and the second layer, the housing being configured to receive a sparge gas from a supply, wherein the sparge gas supplied to the housing is permitted to pass through the first layer and the second layer into a bioprocessing vessel, and wherein the first pore size of the first layer is such that water is not permitted to pass from the bioprocessing vessel past the first layer. In an embodiment, the first layer is gas permeable and water impermeable. In an embodiment, the second layer is gas and water permeable.

In another embodiment of the invention, a bioprocessing system is provided. The bioprocessing system includes a vessel, a flexible bioprocessing bag positionable within the vessel, and a sparger assembly positioned at a bottom of the flexible bioprocessing bag. The sparger assembly includes a first layer having a plurality of pores of a first size, and a second layer disposed above the first layer and having a plurality of holes of a second size, the second size being greater than the first size, wherein the pores of the first layer and the holes of the second layer allow for the passage of a sparge gas through the first layer and the second layer. In an embodiment, the first layer is gas permeable and water impermeable. In an embodiment,the second layer is gas and water permeable. In an embodiment,the first layer is formed form a sintered hydrophobic material. In an embodiment,the second layer is configured as a drilled hole sparging element.

In yet another embodiment of the invention, a sparger assembly is provided. The sparger assembly includes a base layer, a dielectric layer disposed above the base layer, a top layer disposed above the dielectric layer, the top layer having an upper surface, at least one electrode in contact with the dielectric layer, and at least one sparge gas opening in at least the hydrophobic layer for facilitating the formation of a bubble of sparge gas on the upper surface of the top layer for introduction of the sparge gas into a bioreactor vessel. In an embodiment, the top layer is formed from a hydrophobic material. In an embodiment, the at least one electrode is electrically coupled to a voltage source, wherein the voltage source is controllable to supply a voltage to the at least one electrode, and wherein adjusting the voltage supplied to the at least one electrode varies a diameter of the bubble formed on the upper surface of the top layer. In an embodiment, the at least one opening is a plurality of openings including at least a first array of openings and a second array of openings, and the at least one electrode is a plurality of electrodes including at least a first array of electrodes associated with the first array of openings and a second array of electrodes associated with the second array of openings. In an embodiment, the at least one electrode is sandwiched between the base layer and the dielectric layer. [000116] In yet another embodiment, a bioprocessing system is provided. The system includes a vessel, a flexible bioprocessing bag positionable within the vessel, and a sparger assembly positioned at a bottom of the flexible bioprocessing bag. The sparger assembly incudes a base layer, a dielectric layer disposed above the base layer, a top layer disposed above the dielectric layer, the top layer having an upper surface, at least one electrode in contact with the dielectric layer, and at least one sparge gas opening in at least the hydrophobic layer for facilitating the formation of a bubble of sparge gas on the upper surface of the top layer for introduction of the sparge gas into a bioreactor vessel. In an embodiment, the top layer is formed from a hydrophobic material. In an embodiment, the at least one electrode is electrically coupled to a voltage source, wherein the voltage source is controllable to supply a voltage to the at least one electrode, and wherein adjusting the voltage supplied to the at least one electrode varies a diameter of the bubble formed on the upper surface of the top layer. In an embodiment, the at least one electrode is sandwiched between the base layer and the dielectric layer.

In yet another embodiment, a method for bioprocessing is provided. The method includes the steps of positioning a sparger assembly in a bioreactor vessel, the sparger assembly having a base layer, a dielectric layer disposed above the base layer, a top layer disposed above the dielectric layer, the top layer having an upper surface, at least one electrode in contact with the dielectric layer, and at least one sparge gas opening in at least the hydrophobic layer for facilitating the formation of a bubble of sparge gas on the upper surface of the top layer for introduction of the sparge gas into the bioreactor vessel, electrically connecting the at least one electrode to a voltage source, and adjusting a voltage supplied to the at least one electrode to adjust a diameter of the bubble formed on the upper surface of the top layer. In an embodiment, the method further includes fluidly connecting the sparger assembly to a supply of sparge gas. In an embodiment, the method further includes supplying the sparge gas to the sparger assembly to form a first bubble having a first diameter on the upper surface of the top layer, and adjusting a voltage supplied to the at least one electrode to produce a second bubble having a second diameter on the upper surface of the top layer, wherein the first diameter is different from the second diameter. In an embodiment, the at least one opening is a plurality of openings including at least a first array of openings and a second array of openings, and the at least one electrode is a plurality of electrodes including at least a first array of electrodes associated with the first array of openings and a second array of electrodes associated with the second array of openings. The method may further includes supplying a first voltage to the first array of electrodes to produce a plurality of bubbles having a first diameter, and supplying a second voltage that is different from the first voltage to the second array of electrodes to produce a plurality of bubbles having a second diameter that is different from the first diameter.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. [000119] This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A sparger assembly for a bioprocessing system, comprising:
a first layer having a plurality of pores of a first size; and
a second layer disposed above the first layer and having a plurality of holes of a second size, the second size being greater than the first size;
wherein the pores of the first layer and the holes of the second layer allow for the passage of a sparge gas through the first layer and the second layer.

2. The sparger assembly of claim 1, wherein:
the first size is sufficient to allow for passage of gas through the first layer, and to inhibit the passage of water through the first layer.

3. The sparger assembly of claim 1, wherein:
the first layer is hydrophobic and has a three-dimensionally interconnected pore structure.

4. The sparger assembly of claim 1, wherein:
the first layer is formed from a sintered hydrophobic material.

5. The sparger assembly of claim 1, wherein:
the second layer has a plurality of discrete holes through the second layer.

6. The sparger assembly of claim 5, wherein:
the second layer is configured as a drilled hole sparging element.

7. The sparger assembly of claim 1, wherein:
the first size is between about 2 and about 20 micrometers in diameter.

8. The sparger assembly of claim 1, wherein:
the second size is between about 100 and about 500 micrometers in diameter.

9. The sparger assembly of claim 1, further comprising:
a housing receiving the first layer and the second layer, the housing being configured to receive a sparge gas from a supply;
wherein the sparge gas supplied to the housing is permitted to pass through the first layer and the second layer into a bioprocessing vessel; and
wherein the first pore size of the first layer is such that water is not permitted to pass from the bioprocessing vessel past the first layer.

10. The sparger assembly of claim 1, wherein:
the first layer is gas permeable and water impermeable.

11. The sparger assembly of claim 1, wherein:
the second layer is gas and water permeable.

12. A bioprocessing system, comprising:
a vessel;
a flexible bioprocessing bag positionable within the vessel; and
a sparger assembly positioned at a bottom of the flexible bioprocessing bag, the sparger assembly including:
a first layer having a plurality of pores of a first size; and
a second layer disposed above the first layer and having a plurality of holes of a second size, the second size being greater than the first size;
wherein the pores of the first layer and the holes of the second layer allow for the passage of a sparge gas through the first layer and the second layer.

13. The bioprocessing system of claim 12, wherein:
the first layer is gas permeable and water impermeable.

14. The bioprocessing system of claim 12, wherein:
the second layer is gas and water permeable.

15. The bioprocessing system of claim 12, wherein:
the first layer is formed form a sintered hydrophobic material.

16. The bioprocessing system of claim 12, wherein:
the second layer is configured as a drilled hole sparging element.

17. A sparger assembly, comprising:
a base layer;
a dielectric layer disposed above the base layer;
a top layer disposed above the dielectric layer, the top layer having an upper surface;
at least one electrode in contact with the dielectric layer; and
at least one sparge gas opening in at least the hydrophobic layer for facilitating the formation of a bubble of sparge gas on the upper surface of the top layer for introduction of the sparge gas into a bioreactor vessel.

18. The sparger assembly of claim 17, wherein:
the top layer is formed from a hydrophobic material.

19. The sparger assembly of claim 17, wherein:
the at least one electrode is electrically coupled to a voltage source;
wherein the voltage source is controllable to supply a voltage to the at least one electrode; and
wherein adjusting the voltage supplied to the at least one electrode varies a diameter of the bubble formed on the upper surface of the top layer.

20. The sparger assembly of claim 17, wherein:
the at least one sparge gas opening is a plurality of openings including at least a first array of openings and a second array of openings; and
the at least one electrode is a plurality of electrodes including at least a first array of electrodes associated with the first array of openings and a second array of electrodes associated with the second array of openings.

21. The sparger assembly of claim 17, wherein:
the at least one electrode is sandwiched between the base layer and the dielectric layer.

22. A bioprocessing system, comprising:
a vessel;
a flexible bioprocessing bag positionable within the vessel; and
a sparger assembly positioned at a bottom of the flexible bioprocessing bag, the sparger assembly including:
a base layer;
a dielectric layer disposed above the base layer;
a top layer disposed above the dielectric layer, the top layer having an upper surface;
at least one electrode in contact with the dielectric layer; and
at least one sparge gas opening in at least the hydrophobic layer for facilitating the formation of a bubble of sparge gas on the upper surface of the top layer for introduction of the sparge gas into a bioreactor vessel.

23. The bioprocessing system of claim 22, wherein:
the top layer is formed from a hydrophobic material.

24. The bioprocessing system of claim 22, wherein:
the at least one electrode is electrically coupled to a voltage source;
wherein the voltage source is controllable to supply a voltage to the at least one electrode; and
wherein adjusting the voltage supplied to the at least one electrode varies a diameter of the bubble formed on the upper surface of the top layer.

25. The bioprocessing system of claim 22, wherein:
the at least one electrode is sandwiched between the base layer and the dielectric layer.

26. A method for bioprocessing, comprising the steps of:
positioning a sparger assembly in a bioreactor vessel, the sparger assembly having a base layer, a dielectric layer disposed above the base layer, a top layer disposed above the dielectric layer, the top layer having an upper surface, at least one electrode in contact with the dielectric layer, and at least one sparge gas opening in at least the hydrophobic layer for facilitating the formation of a bubble of sparge gas on the upper surface of the top layer for introduction of the sparge gas into the bioreactor vessel;
electrically connecting the at least one electrode to a voltage source; and
adjusting a voltage supplied to the at least one electrode to adjust a diameter of the bubble formed on the upper surface of the top layer.

27. The method according to claim 26, further comprising the step of:
fluidly connecting the sparger assembly to a supply of sparge gas.

28. The method according to claim 26, further comprising the steps of:
supplying the sparge gas to the sparger assembly to form a first bubble having a first diameter on the upper surface of the top layer; and
adjusting a voltage supplied to the at least one electrode to produce a second bubble having a second diameter on the upper surface of the top layer;
wherein the first diameter is different from the second diameter.

29. The method according to claim 26, wherein:
the at least one opening is a plurality of openings including at least a first array of openings and a second array of openings; and
the at least one electrode is a plurality of electrodes including at least a first array of electrodes associated with the first array of openings and a second array of electrodes associated with the second array of openings; and
wherein the method further includes supplying a first voltage to the first array of electrodes to produce a plurality of bubbles having a first diameter, and supplying a second voltage that is different from the first voltage to the second array of electrodes to produce a plurality of bubbles having a second diameter that is different from the first diameter.
